(19) United States
(12) Patent Application Publication
(10) Pub. No.: US 2023/0272431 A1
(43) Pub. Date: Aug. 31, 2023

(54) METHODS AND COMPOSITIONS FOR EDITING THE B2M LOCUS IN B CELLS

(71) Applicant: SEATTLE CHILDREN'S HOSPITAL (DBA SEATTLE CHILDREN'S RESEARCH INSTITUTE), Seattle, WA (US)

(72) Inventors: David J. Rawlings, Seattle, WA (US); Richard G. James, Seattle, WA (US); Claire Marie Stoffers, Seattle, WA (US); Peter J. Cook, Seattle, WA (US)

(21) Appl. No.: 18/012,031

(22) PCT Filed: Jun. 30, 2021
(86) PCT No.: PCT/US21/39953

§ 371 Date: Dec. 21, 2022

Related U.S. Application Data
(60) Provisional application No. 63/047,978, filed on Jul. 3, 2020.
(60) Provisional application No. 63/114,431, filed on Nov. 16, 2020.

(51) Int. Cl. C12N 15/90; C12N 15/11; C12N 9/22; C12N 5/0781; A61K 35/17

(52) U.S. Cl. CPC C12N 15/907; C12N 15/11; C12N 9/22; C12N 5/0635; A61K 35/17; C12N 2310/20; C12N 2800/80

(57) ABSTRACT

Some embodiments of the methods and compositions provided herein include preparing modified B cells. In some embodiments, an endogenous beta-2 microglobulin (B2M) gene in a B cell is modified. Some embodiments relate to increasing the resistance of modified B cells to killing by allogeneic immune cells. In some embodiments, the endogenous B2M gene is inactivated increasing the resistance of the modified B cell to killing by allogeneic immune cells. In some embodiments, a replacement MHC-I is inserted into an inactivated endogenous B2M gene increasing the resistance of the modified B cell to killing by allogeneic immune cells. Some embodiments include enriching for successfully modified cells.

### CROSS-REFERENCE TO RELATED APPLICATIONS

This application claims priority to U.S. Prov. App. No. 63/114,131 filed Nov. 16, 2020 entitled “METHODS AND COMPOSITIONS FOR EDITING THE B2M LOCUS IN B CELLS”, and to U.S. Prov. App. No. 63/047,978 filed Jul. 3, 2020 entitled “METHODS AND COMPOSITIONS FOR EDITING THE B2M LOCUS IN B CELLS” which are each expressly incorporated by reference in its entirety.

### REFERENCE TO SEQUENCE LISTING

The present application is being filed along with a Sequence Listing in electronic format. The Sequence Listing is provided as a file entitled SCRI295WOSEQLIST, created Jun. 29, 2021, which is approximately 4.5 Kb in size. The information in the electronic format of the Sequence Listing is incorporated herein by reference in its entirety.

### FIELD OF THE INVENTION

Some embodiments of the methods and compositions provided herein include preparing modified B cells. In some embodiments, an endogenous beta-2 microglobulin (B2M) gene in a B cell is modified. Some embodiments relate to increasing the resistance of modified B cells to killing by allogeneic immune cells. In some embodiments, the endogenous B2M gene is inactivated increasing the resistance of the modified B cell to killing by allogeneic immune cells. In some embodiments, a replacement MHC-I is inserted into an inactivated endogenous B2M gene increasing the resistance of the modified B cell to killing by allogeneic immune cells. Some embodiments include enriching for successfully modified cells.

### BACKGROUND OF THE INVENTION

Genome editing applications have increased in frequency as a result of the efficacy and ease of use of recent tools, e.g., CRISPR and TALEN systems. However, genome editing in clinically relevant human somatic cells remains a challenge, for example, due to unwanted host immune responses to allogeneic transplantation of such cells. Accordingly, there exists a need for cells suitable for allogeneic transplantation that eliminate or reduce the likelihood of triggering unwanted recipient immune responses to allogeneic transplants of such cells.

### SUMMARY OF THE INVENTION

Some embodiments of the methods and compositions provided herein include a system for modifying an endogenous beta-2 microglobulin (B2M) gene in a cell, comprising: a nuclease capable of cleaving a targeted locus in an endogenous B2M gene in a genome of the cell, or a nucleic acid encoding the nuclease; and optionally: a guide RNA (gRNA) comprising a sequence complementary to the B2M gene, and/or a repair template comprising a first homology arm, a second homology arm, and a nucleic acid encoding a payload therebetween, wherein the first homology arm and/or the second homology arm has homology to a sequence in the B2M gene.

In some embodiments, the gRNA comprises: a nucleotide sequence having at least 85%, 86%, 87%, 88%, 89%, 90%, 95%, 96%, 97%, 98%, 99%, or 100%, sequence identity with the nucleotide sequence of any one of SEQ ID NOs: 01-04; a nucleotide sequence of any one of SEQ ID NOs: 01-04; or a variant thereof having no more than 3 mismatches compared to any one of SEQ ID NOs: 01-04.

In some embodiments, the gRNA is adapted to inactivate the endogenous B2M gene.

In some embodiments, the targeted locus is within a first coding exon of the B2M gene.

In some embodiments, the nuclease comprises a Cas nuclease. In some embodiments, the Cas nuclease comprises a Cas9 nuclease.

In some embodiments, the nuclease is selected from the group consisting of a zinc finger nuclease, a transcription activator-like effector nuclease (TALEN), a homing endonuclease (HEs), and a combined TALEN-HE protein (megaTALs).

In some embodiments, the repair template lacks a sequence targeted by the nuclease, and/or lacks a sequence capable of hybridizing to a sequence of the gRNA or complement thereof. In some embodiments, the payload lacks the sequence targeted by the nuclease, and/or lacks a sequence capable of hybridizing to a sequence of the gRNA or complement thereof. In some embodiments, the first arm of homology and/or the second arm of homology lack the sequence targeted by the nuclease, and/or lack a sequence capable of hybridizing to a sequence of the gRNA or complement thereof.

In some embodiments, the payload comprises a first nucleic acid encoding a first polypeptide. In some embodiments, the payload comprises a second nucleic acid encoding a second polypeptide. In some embodiments, a nucleic acid encoding a ribosome skip sequence is located between the first nucleic acid and the second nucleic acid.

In some embodiments, the first polypeptide or second polypeptide encodes a B2M cDNA. In some embodiments, the first polypeptide or second polypeptide encodes a non-polymeric HLA polypeptide, or a therapeutic polypeptide. In some embodiments, the non-polymeric HLA polypeptide is selected from HLA-E, HLA-F, or HLA-G. Some embodiments also include a third polypeptide encoding a self-peptide to be presented by the non-polymeric HLA polypeptide In some embodiments, the therapeutic polypeptide comprises an enzyme, an antibody or antigen binding fragment thereof, a receptor, a chimeric antigen receptor, or a cytokine. In some embodiments, the therapeutic polypeptide comprises factor IX, angiotensin-converting enzyme 2 (Ace2), beta-glucocerebrosidase (GBA), alpha-galactosidase A (GLA), or acid alpha-glucosidase (GAA).

In some embodiments, the first nucleic acid is operably linked to a promoter of the endogenous B2M gene.

In some embodiments, the first polypeptide is in-frame with a polypeptide encoded by the endogenous B2M gene.

In some embodiments, the payload comprises a heterologous promoter, and the first nucleic acid is operably linked to the heterologous promoter. In some embodiments, the heterologous promoter is a constitutive promoter. In some embodiments, the promoter selected from an MND promoter, and an EF1 alpha promoter, or a PGK promoter.

In some embodiments, a vector comprises the repair template. In some embodiments, the vector comprises a viral vector. In some embodiments, the viral vector comprises an adeno-associated virus (AAV) vector, or a lentiviral vector.

Some embodiments also include a cell, wherein the cell comprises the nuclease; and optionally, the gRNA, and/or repair template. In some embodiments, the cell comprises the nuclease. In some embodiments, the cell comprises the gRNA, and/or repair template. In some embodiments, the cell is selected from the group consisting of a B cell, a hematopoietic stem cell, an early pro-B cell, a late pro-B cells, a large pre-B cell, a small pre-B cell, an immature B cell, a T1 B cell, a T2 B cell, a marginal zone B cell, a mature B cell, a naïve B cell, a plasmablast (short lived) cell, a GC B cell, a memory B cell, and a long lived plasma cell. In some embodiments, the cell is a B cell. In some embodiments, the cell is human.

In some embodiments, the cell is autologous to a subject.

In some embodiments, the cell is allogeneic to a subject.

In some embodiments, the cell is ex vivo.

In some embodiments, the cell is in vivo.

Some embodiments include a pharmaceutical composition comprising any one of the systems provided herein and a pharmaceutically acceptable excipient.

Some embodiments include a method for preparing a modified cell, comprising obtaining any one of the systems provided herein; introducing the nuclease into a first cell; and optionally: introducing the gRNA into the cell, and/or introducing the repair template into the cell, thereby preparing a modified cell comprising a modified B2M locus in the genome of the modified cell.

In some embodiments, the modified B2M locus comprises an inactivated B2M endogenous gene. In some embodiments, the gRNA is adapted to inactivate the endogenous B2M gene. Some embodiments also include selecting for the modified cell. In some embodiments, the selecting comprising contacting the modified cell with an immune cell. In some embodiments, the immune cell is allogeneic to the first cell. In some embodiments, the immune cell comprises an MHC-I different from an MHC-I of the first cell. In some embodiments, the immune cell is selected from a T cell, such as a cytotoxic CD8+ T cell, or a natural killer cell.

In some embodiments, the immune cell is in vivo.

In some embodiments, the immune cell is ex vivo.

In some embodiments, the modified B2M locus expresses an active B2M gene or B2M cDNA. In some embodiments, the repair template lacks a sequence targeted by the nuclease, and/or lacks a sequence capable of hybridizing to a sequence of the gRNA or complement thereof, such that the modified B2M locus lacks the sequence targeted by the nuclease, and/or lacks the sequence capable of hybridizing to a sequence of the gRNA or complement thereof. In some embodiments, the repair template comprises a B2M cDNA. Some embodiments also include selecting for the modified cell. In some embodiments, the selecting comprising contacting the modified cell with an immune cell. In some embodiments, the immune cell is autologous to the first cell. In some embodiments, the immune cell is selected from a T cell, such as a cytotoxic CD8+ T cell, or a natural killer cell.

In some embodiments, the immune cell is in vivo.

In some embodiments, the immune cell is ex vivo.

In some embodiments, the modified B2M locus expresses a replacement MHC-I. In some embodiments, the modified B2M locus comprises an inactivated B2M endogenous gene. In some embodiments, the repair template comprises a payload encoding a B2M cDNA, a non-polymeric HLA polypeptide, and/or a self-peptide to be presented by the non-polymeric HLA polypeptide. Some embodiments also include selecting for the modified cell. In some embodiments, the selecting comprising contacting the modified cell with an immune cell. In some embodiments, the immune cell is allogeneic to the first cell. In some embodiments, the immune cell is selected from a T cell, such as a cytotoxic CD8+ T cell, or a natural killer cell.

In some embodiments, the immune cell is in vivo.

In some embodiments, the immune cell is ex vivo.

In some embodiments, the first cell is a B cell.

Some embodiments include a cell prepared by any one of the methods provided herein.

Some embodiments include a method for enriching for a modified B cell having increased resistance to killing by a T cell or natural killer cell, comprising: preparing a modified cell by any one of the methods provided herein, wherein the first cell is a B cell, and wherein the modified B2M locus is inactivated; and contacting the modified cell with a T cell or natural killer cell, wherein the T cell or natural killer cell is allogeneic to the first cell, and wherein the modified B cell has an increased resistance to killing by the T cell or natural killer cell compared to a B cell expressing a B2M gene.

Some embodiments include a method for enriching for a modified B cell, comprising preparing a modified cell by any one of the methods provided herein, wherein the modified B2M locus expresses an active B2M gene or B2M cDNA; and contacting the modified cell with a T cell or natural killer cell, wherein the T cell or natural killer cell is autologous to the first cell, and wherein a cell not expressing an active B2M gene or B2M cDNA is killed by the T cell or natural killer cell.

Some embodiments include a method for enriching for a modified B cell, comprising preparing a modified cell by any one of the methods provided herein, wherein the modified B2M locus expresses a replacement MHC-I, wherein the repair template comprises a payload encoding a B2M cDNA, a non-polymeric HLA polypeptide, and a self-peptide to be presented by the non-polymeric HLA polypeptide; and contacting the modified cell with a T cell or natural killer cell, wherein the T cell or natural killer cell is allogenic to the first cell.

Some embodiments include a method for preparing a modified B cell for an allogeneic infusion, comprising preparing a modified cell by any one of the methods provided herein, wherein the cell is a B cell and the repair template comprises a payload encoding a B2M cDNA, a non-polymeric HLA polypeptide, and a self-peptide to be presented by the non-polymeric HLA polypeptide. Some embodiments also include administering the modified B cell to a subject, wherein the B cell is allogeneic to the subject. Tn some embodiments, the non-polymeric HLA polypeptide is selected from HLA-E, HLA-F, or HLA-G. In some embodiments, the subject is mammalian. In some embodiments, the subject is human.

Some embodiments of the methods and compositions provided herein include a method for preparing a modified B cell, comprising: inactivating an endogenous beta-2 microglobulin (B2M) gene in a B cell; and introducing a polynucleotide encoding a fusion protein into the inactivated B2M gene thereby obtaining a modified B cell, wherein the fusion protein comprises an exogenous B2M polypeptide.

Some embodiments of the methods and compositions provided herein include a method for in vivo enrichment of a modified B cell in an autologous subject, comprising: inactivating an endogenous beta-2 microglobulin (B2M) gene in a B cell; introducing a polynucleotide encoding a fusion protein into the inactivated B2M gene thereby obtaining a modified B cell, wherein the fusion protein comprises an exogenous B2M polypeptide; and administering the modified B cell to a subject, wherein the B cell is autologous to the subject.

Some embodiments of the methods and compositions provided herein include a method for preparing a modified B cell for an allogeneic infusion, comprising: inactivating an endogenous beta-2 microglobulin (B2M) gene in a B cell, and introducing a polynucleotide encoding a fusion protein into the inactivated B2M gene thereby obtaining a modified B cell, wherein the fusion protein comprises: an exogenous B2M polypeptide, a non-polymeric HLA polypeptide, and a self-peptide to be presented by the non-polymeric HLA polypeptide. Some embodiments also include administering the modified B cell to a subject, wherein the B cell is allogeneic to the subject. In some embodiments, the non-polymeric HLA polypeptide is selected from HLA-E or HLA-G.

In some embodiments, the endogenous B2M gene is inactivated by an insertion, deletion or substitution of at least a portion of the endogenous B2M gene.

In some embodiments, the inactivating an endogenous B2M gene comprises introducing into the B cell a clustered regularly interspersed short palindromic repeat DNA (CRISPR) coupled to a Cas nuclease. In some embodiments, the Cas nuclease comprises a Cas9 nuclease. In some embodiments, the inactivating an endogenous B2M gene comprises introducing a guide RNA (gRNA) into the B cell. In some embodiments, the gRNA comprises a nucleotide sequence having at least 90%, 91%, 92%, 93%, 94%, 95%, 96%, 97%, 98%, or 99% sequence identity with the nucleotide sequence of any one of SEQ ID NOs:01-04.

In some embodiments, the inactivating an endogenous B2M gene comprises introducing into the B cell a viral vector comprising a repair template. In some embodiments, the viral vector comprises an adeno-associated virus (AAV) vector.

In some embodiments, the inactivating an endogenous B2M gene comprises introducing a nuclease into the B cell, wherein the nuclease is selected from the group consisting of a zinc finger nuclease, a transcription activator-like effector nuclease (TALEN), a homing endonuclease (HE), and a combined TALEN-HE protein (megaTAL)

In some embodiments, the polynucleotide encoding a fusion protein is introduced into the inactivated B2M gene such that the fusion protein is in-frame with an exon of the endogenous B2M gene.

In some embodiments, the polynucleotide comprises a homology arm, a nucleic acid encoding a self-cleaving peptide, and/or a promoter.

In some embodiments, the introducing a polynucleotide encoding a fusion protein into the inactivated B2M gene introducing into the B cell a clustered regularly interspersed short palindromic repeat DNA (CRISPR) coupled to a Cas nuclease. In some embodiments, the Cas nuclease comprises a Cas9 nuclease. In some embodiments, the introducing a polynucleotide encoding a fusion protein into the inactivated B2M gene comprises introducing a guide RNA (gRNA) into the B cell. In some embodiments, the gRNA comprises a nucleotide sequence having at least 90%, 91%, 92%, 93%, 94%, 95%, 96%, 97%, 98%, or 99% sequence identity with the nucleotide sequence of any one of SEQ ID NOs:01-04.

In some embodiments, the introducing a polynucleotide encoding a fusion protein into the inactivated B2M gene comprises introducing into the B cell a viral vector comprising a repair template. In some embodiments, the viral vector comprises an adeno-associated virus (AAV) vector.

In some embodiments, the introducing a polynucleotide encoding a fusion protein into the inactivated B2M gene comprises introducing a nuclease into the B cell, wherein the nuclease is selected from the group consisting of a zinc finger nuclease, a transcription activator-like effector nuclease (TALEN), a homing endonuclease (HE), and a combined TALEN-HE protein (megaTAL).

In some embodiments, the inactivating an endogenous beta-2 microglobulin (B2M) gene in a B cell; and the introducing a polynucleotide encoding a fusion protein into the inactivated B2M gene are performed sequentially. In some embodiments, the inactivating an endogenous beta-2 microglobulin (B2M) gene in a B cell; and the introducing a polynucleotide encoding a fusion protein into the inactivated B2M gene are performed concurrently.

In some embodiments, the fusion protein comprises a therapeutic polypeptide or a therapeutic nucleic acid. In some embodiments, the therapeutic polypeptide comprises an enzyme, an antibody or antigen binding fragment thereof, a receptor, a chimeric antigen receptor, or a cytokine. In some embodiments, the therapeutic polypeptide comprises factor IX, angiotensin-converting enzyme 2 (Ace2), beta-glucocerebrosidase (GBA), alpha-galactosidase A (GLA), or acid alpha-glucosidase (GAA).

In some embodiments, the B cell is selected from the group consisting of a hematopoietic stem cell, an early pro-B cell, a late pro-B cells, a large pre-B cell, a small pre-B cell, an immature B cell, a T1 B cell, a T2 B cell, a marginal zone B cell, a mature B cell, a naïve B cell, a plasmablast (short lived) cell, a GC B cell, a memory B cell, and a long lived plasma cell.

In some embodiments, the subject is mammalian. In some embodiments, the subject is human.

Some embodiments of the methods and compositions provided herein include a modified B cell prepared by any one of the foregoing methods.

Some embodiments of the methods and compositions provided herein include a pharmaceutical composition comprising any one of the modified B cells provided herein, and a pharmaceutically acceptable carrier.

Some embodiments of the methods and compositions provided herein include an isolated nucleic acid comprising a nucleotide sequence having at least 90%/6, 91%, 92%, 93%, 94%, 95%, 96%, 97%, 98%, or 99% sequence identity with the nucleotide sequence of any one of SEQ ID NOs:01-04.

### BRIEF DESCRIPTION OF THE DRAWINGS

FIG. 1 depicts a flow analysis of MHC-II expression in plasma cells (CD138+) cultured under conditions which included: (1) Activation for 2 days with MCD40L and either CpG B or αCD180; (2) Phase I for 3 days with IL-21 and either IL-6 or R848; and (3) Phase II for 3 days with IL-21.

FIG. 2 depicts a schematic of a murine B2M locus and includes a B2M gene, with corresponding B2M mRNA depicting exons, and corresponding protein coding portions of the mRNA depicted as a B2M cDNA; also shown are sites for guide RNAs: gRNA-1 and gRNA_5.

FIG. 3 depicts a graph showing deletion frequency (ICE score) and predicted inactivation of the B2M gene (knockout (KO) score) for guide RNAs: gRNA-1 and gRNA_5, and their reverse sequences.

FIG. 4A depicts a flow analysis murine B cells treated with gRNA-1, gRNA_5, or control.

FIG. 4B depicts a bar chart for percentage H2Kb protein expressing cells for cells treated with gRNA-1, gRNA 5, or control.

FIG. 4C depicts a flow analysis human B cells treated with gRNAs to target the human B2M locus (gRNA-1, gRNA-2), or control.

FIG. 5A depicts cytotoxic CD8+ T cells expressing TCRs proteins that recognize mismatched MHC polypeptides killing cells with mismatched MHC-I proteins (left panel); and, cells lacking MHC-I may not induce killing by the cytotoxic CD8+ T cells (right panel).

FIG. 5B depicts a mixed cell assay to determine if B cells lacking MHC-I would be resistant to killing by such cytotoxic CD8+ T-Cells with T cells from BALB/c mice (HLA, class D) and B cells from C57BL/6 mice (HLA, class B).

FIG. 6A depicts a flow analysis for mixed cultures of B cells treated with B2M gRNA_1 RNP, Rosa26RNP, or mock, and T cells.

FIG. 6B depicts a graph of a ratio of CD45.1 (B2M knockout B cells) to CD45.2 (control B cells) ratios compared to a ratio of T cells to B cells for mixed cultures.

FIG. 7 depicts a timeline for a CD8+ in vitro killing assay.

FIG. 8A depicts a FACS analysis of modified cells for CD45.2 (Target) and CD45.1 (Decoy) C57/B6 B-Cells at 24 h and 48 h Balb/C CD8+ T-Cell co-culture time points.

FIG. 8B depicts lines graphs for modified cells relating to quantitation of the CD45.1CD45.2 ratio from the experiment related to FIG. 8A, normalized to the base ratio in co-culture conditions with no CD8+ T-Cells.

FIG. 9A depicts a FACS analysis of modified cells for H2Kb (the relevant MHC1 haplotype in C57/B6 B-Cells) within the CD45.1 B-Cell population in the experiment related to FIG. 8A.

FIG. 9B depicts lines graphs for modified cells relating to quantitation of H2Kb knockout level in B2M edited CD45.1 B-Cells at 24 h and 48 h time points with increasing CD8+ T-Cell ratios.

FIG. 10 depicts a schematic of a construct (3307) to modify the B2M locus and includes sequences encoding: 5′ and 3′ homology arms (HA), an MND promoter operably linked to a GFP polypeptide, a P2A self-cleaving peptide, and a B2M cDNA.

FIG. 11 is a flow analysis of primary mouse B cells edited using a combination of B2M gRNA_1 RNP delivery and 3307 AAV transduction.

FIG. 12A depicts a strategy for a substitution at the B2M locus in which the B2M locus is modified to inactivate endogenous B2M expression, and to express an engineered HLA-E.

FIG. 12B depicts a mixed cell assay to test B cell survival of a B cell modified to inactivate endogenous B2M expression, and to express an engineered HLA-E.

FIG. 13 depicts an HDR donor template (3310) encoding 5′ and 3′ homology arms (HA); an exogenous MND promoter; a fusion protein including a Qdm self-peptide, a B2M cDNA, and a non-polymorphic HLA-E alpha chain connected via flexible linkers; a 2A self-cleaving peptide; a GFP marker; and a SV40 polyadenylation sequence.

FIG. 14 is a flow analysis of B cells edited with the construct depicted in FIG. 13 and for GFP expression in H2Kb−, differentiated B cells at day 7.

FIG. 15A depicts a study to establish parameters for NK cell killing with in vivo engraftment of MHC-I knockout cells.

FIG. 15B depicts a study for a mouse model for allogeneic B cell engraftment with murine cells.

FIG. 16 depicts a study for a xenograft model for allogeneic B cell engraftment with human cells.

### DETAILED DESCRIPTION

Some embodiments of the methods and compositions provided herein include preparing modified B cells. In some embodiments, an endogenous beta-2 microglobulin (B2M) gene in a B cell is modified. Some embodiments relate to increasing the resistance of modified B cells to killing by allogeneic immune cells. In some embodiments, the endogenous B2M gene is inactivated increasing the resistance of the modified B cell to killing by allogeneic immune cells. In some embodiments, a replacement MHC-1 is inserted into an inactivated endogenous B2M gene increasing the resistance of the modified B cell to killing by allogeneic immune cells. Some embodiments include enriching for successfully modified cells.

Some embodiments of the methods and compositions provided herein include preparing modified B cells. In some embodiments, an endogenous beta-2 microglobulin (B2M) gene in a B cell is modified. In some embodiments, the endogenous B2M gene in a B cell is inactivated. In some such embodiments, a polynucleotide encoding a fusion protein is introduced into the inactivated B2M gene. Some embodiments include the preparation and use of B cells modified at the B2M locus for allogeneic infusions, expression of fusion proteins, and/or in vivo enrichment of modified cells.

Some embodiments provided herein include methods for introduction of exogenous transgenes and engineering genomic DNA at the B2M locus for the purposes of improving delivery of a B cell therapy. Some embodiments include use of CRISPR-Cas9 recombinant nucleoprotein complexes (guide RNA and protein) with AAV delivered repair templates. Some embodiments include use of an alternate nuclease (e.g., zinc finger nuclease, talen, or megatalen) or repair template (e.g., single or double-stranded DNA) platforms for gene delivery. In some embodiments, sequences that are delivered to the B2M locus facilitate allogenic delivery and/or enrichment of engineered human B cells. In some embodiments, sequences that are delivered to the B2M locus facilitate autologous delivery by enrichment of engineered B cells in vivo.

Some embodiments include synthetic guide RNAs for human B2M useful to efficiently edit the B2M locus, for example to knock-down/inactivate MHC-T. Some embodiments include repair templates for introduction of exogenous sequence into B2M, while also maintaining expression of endogenous B2M. Such repair templates can include sequences encoding therapeutic proteins such as factor IX, angiotensin-converting enzyme 2 (Ace2), beta-glucocerebrosidase (GBA), alpha-galactosidase A (GLA), or acid alpha-glucosidase (GAA). Some embodiments include repair templates that replace endogenous B2M with B2M fused to non-polymeric versions of HLA (e.g., HLA-E, or HLA-G) and peptides that inhibit NK cell killing. These sequences facilitate allogeneic engraftment of plasma cells without graft rejection by host immune cells (e.g., T and/or NK cells). Some embodiments include a repair template that replaces B2M with B2M fused to non-polymorphic HLA and introduces exogenous sequences for therapeutic proteins.

Some embodiments relate to increasing the resistance of modified B cells to killing by allogeneic immune cells. For example, in some embodiments, the endogenous B2M gene is inactivated increasing the resistance of the modified B cell to killing by allogeneic immune cells. Some such embodiments include compositions and methods for enriching for a modified B cell having increased resistance to killing by an allogeneic T cell or allogeneic natural killer cell. Some such embodiments include preparing a modified B cell, wherein the B2M locus of the cell's genome is inactivated, and contacting the modified cell with an immune cell, such as a T cell, such as a CD8+ T cell, wherein the immune cell is allogeneic to the modified B cell. In some such embodiments, the lack of a mismatched MHC-I between the modified B cell the immune cell increases the resistance of the modified B cell to killing by the immune cell.

Some embodiments relate to increasing the resistance of modified B cells to killing by allogeneic immune cells. For example, in some embodiments, a replacement MHC-I is inserted into an inactivated endogenous B2M gene increasing the resistance of the modified B cell to killing by allogeneic immune cells. Some such embodiments include compositions and methods for enriching for a modified B cell having increased resistance to killing by an allogeneic T cell or allogeneic natural killer cell by replacing an MHC-I of a cell. Some such embodiments include preparing a modified B cell, wherein the modified B2M locus expresses a replacement MHC-I. In some such embodiments, the endogenous B2M locus is inactivated and replaced by a payload encoding a B2M cDNA, a non-polymeric HLA polypeptide, and a self-peptide to be presented by the non-polymeric HLA polypeptide. Some embodiments also include contacting the modified cell with an immune cell, such as T cell, such as a CD8+ T cell, wherein the immune cell is allogenic to the immune cell.

Some such embodiments include compositions and methods for enriching for a modified B cell having certain successful modifications. Some such embodiments include preparing a modified B cell, wherein the modified B2M locus of the cell expresses an active B2M gene or B2M cDNA, and contacting the modified cell with an immune cell, such as a T cell, such as a CD8+ T cell, wherein the immune is autologous to the modified B cell. Modified cells in which modifications do not result in successful expression of an active B2M gene or B2M cDNA can be killed, removed, or cleared by the immune cell.

Certain aspects useful with certain embodiments of the methods and compositions provided herein are disclosed in U.S. 20180141992; Hung K. L. et al., (2018) Mol Ther 26:456-467; Voss J. E. et al., (2019) Elife 8:e42995; Hartweger H., et al., (2019) J Exp Med 216:1301-1310; Johnson M. J. et al., (2018) Scientific Reports 8:12144; and Moffett H. F. et al., (2019) Sci Immunol 4(35) which are each expressly incorporated by reference in its entirety.

### Definitions

As used herein, “genome editing” has its plain and ordinary meaning when read in light of the specification, and may include but is not limited to, for example, a process that include methods for genetic engineering in which DNA is inserted, deleted or replaced in the genome of a living organism. Editing a gene is also known as gene editing. In some alternatives described herein, a method of making plasma cells or plasma cell precursors that express a molecule, such as a macromolecule is provided, in which B cells or B cell precursors are subjected to at least one round of genome editing. Methods of genome editing can include, but is not limited to nucleic acid being inserted, deleted or replaced in the genome of a cell. In some alternatives, a nuclease is used to achieve this process. In some alternatives, the nuclease is engineered. In some alternatives, the methods include inducing double strand breaks that are repaired through nonhomologous end-joining (NHEJ) or homologous recombination (HR). In some alternatives, the step of genome editing is performed by introduction of a single stranded nucleic acid. In some alternatives, the at least one round of genome editing further comprises cycling the B-cells for homologous recombination of the single stranded DNA oligonucleotides or recombinant adeno-associated virus into the candidate genetic loci. In some alternatives, the genome editing of the B cells for protein expression is performed in the absence of viral integration. In some alternatives, a second round of genome editing is performed to excise a region. In some alternatives, a third round of genome editing is performed to result in expression of a drug activatable growth enhancer. In some alternatives herein, the genome editing is performed by nonpathogenic AAV mediated editing by direct homologous recombination.

Genome editing can also employ the use of RNA and protein-based transfection. For example, the CRISPR/Cas system can be modified to edit genomes. This technique requires the delivery of the Cas nuclease complexed with a synthetic guide RNA (gRNA) into a cell, thus the cell's genome can be cut at a specific location and allow existing genes to be removed and/or add new ones. Thus, CRISPR/Cas and related programmable endonuclease systems are rapidly becoming significant genome editing tools of the biomedical research laboratory, with their application for gene disruption and/or gene targeting as demonstrated in a variety of cultured cell and model organism systems. In some alternatives, of the CRISPR/Cas system described herein, the Cas nuclease comprises Cas1, Cas2, Cas3, Cas4, Cas5, Cas6, Cas7, Cas8 or Cas9.

The basic components of CRISPR/Cas system comprise a target gene, a protospacer adjacent motif (PAM), a guide RNA, Cas endonuclease. An important aspect of applying CRISPR/Cas for genome editing is the need for a system to deliver the guide RNAs efficiently to a wide variety of cell types. This could, for example, involve delivery of an in vitro generated guide RNA as a nucleic acid (the guide RNA generated by in vitro transcription or chemical synthesis). In some alternatives, the nucleic acid could be rendered nuclease resistant by incorporation of modified bases.

The CRISPR-Cas system falls into two classes. The Class 1 system has a complex of multiple Cas proteins for the degradation of foreign nucleic acids. The Class 2 system has a single large Cas protein for a same purpose for the degradation of foreign nucleic acids. There are a 93 cas genes that are grouped into 35 families. 11 of the 35 families from a cas core which includes the protein families CAS1 to CAS9. As described herein, Cas comprises Cas1, Cas2, Cas3, Cas4, Cas5, Cas6, Cas7, Cas8 or Cas9.

Gene editing may also be performed by a novel non-nuclease-based gene editing platform. A novel family of AAVs were previously isolated from human hematopoietic stem cells. These nonpathogenic AAVs are naturally present in healthy individuals and may possess unique gene editing and gene transfer properties. This technique is also described as AAV mediated editing by direct homologous recombination (AmENDR™). This process is homologous recombination by a natural biological mechanism that is used by cells to ensure highly precise DNA repair.

AAV mediated editing by direct homologous recombination is initiated by design of homology sequence “arms” that are specific to a region of the genome and results in a permanent correction in the DNA when administered to cells. In some alternatives herein, the gene editing is performed by nonpathogenic AAV mediated editing by direct homologous recombination. The identification of novel AAV genomes are described in Smith et al. (Mol Ther. 2014 September; 22(9): 1625-1634; incorporated by reference in its entirety herein). The novel AAVs described by Smith et al., represents a new class of genetic vector for the manipulation of HSC genomes. Furthermore, these vectors may greatly expand the ability to deliver genes to targeted tissues and cells including cells that are refractory to gene transfer which circumventing prevalent preexisting immunity to AAV2. In some alternatives, the gene editing is performed by nonpathogenic AAVs naturally present in hematopoietic cells, wherein the editing is performed by AAV mediated editing by direct homologous recombination using the nonpathogenic AAVs as described in Smith et al.

As used herein, “engineered nucleases” have their plain and ordinary meaning when read in light of the specification, and may include but is not limited to, for example, enzymes that are engineered to be hybrid enzymes which can be used to specifically recognize a DNA sequence and efficiently edit the genome by the introduction of double-strand breaks. Without being limiting, there are four families of preferred engineered nucleases suitable for use with embodiments described herein including meganucleases, zinc finger nucleases (ZFN), transcription activator like effector-based nucleases (TALEN), or the CRISPR-Cas system.

As used herein, “meganucleases” have their plain and ordinary meaning when read in light of the specification, and may include but is not limited to, for example, endodeoxyribonucleases characterized by a large recognition site (double-stranded DNA sequences of 12 to 40 base pairs). In some alternative methods for making a plasma cell or plasma cell precursor that expresses a molecule such as a macromolecule, the method comprises: (a) isolating B cells, (b) developing the B cells, (c) performing a first round of genome editing of the B cells for protein expression in absence of viral integration, (d) expanding the B cells, and (e) differentiating the B cells, optionally, after step (c) or (d), thereby producing plasma cells that express a protein. In some alternatives, the first round of genome editing is performed by an RNA and protein-based transfection. In some alternatives, the nuclease is a meganuclease.

As used herein, “zinc finger nucleases (ZFN)” have their plain and ordinary meaning when read in light of the specification, and may include but is not limited to, for example, engineered restriction enzymes that are generated by fusing a zinc finger DNA-binding domain to a DNA-cleavage domain. Zinc finger domains can be engineered to target specific desired DNA sequences and this enables zinc-finger nucleases to target unique sequences within complex genomes. In some alternative methods for making a plasma cell that expresses a molecule, such as a macromolecule, the method comprises: (a) isolating B cells, (b) developing the B cells, (c) performing a first round of genome editing of the B cells for protein expression in absence of viral integration, (d) expanding the B cells, and (e) differentiating the B cells, optionally, after step (c) or (d), thereby producing plasma cells that express a protein. In some alternatives, the first round of genome editing is performed by an RNA and protein-based transfection. In some alternatives, the nuclease is a zinc finger nuclease.

As used herein, “transcription activator-like effector nucleases,” (TALEN), have their plain and ordinary meaning when read in light of the specification, and may include but is not limited to, for example, restriction enzymes that can be engineered to cut specific sequences or sites in DNA. They are made by fusing a TAL effector DNA-binding domain to a DNA cleavage domain (a nuclease which cuts DNA strands). Transcription activator-like effectors (TALEs) can be engineered to bind to a desired DNA sequence, so when combined with a nuclease, the DNA can be cut at specific locations. Thus, the restriction enzymes can be introduced into cells, for use in genome editing or for genome editing in situ, a technique known as genome editing with engineered nucleases. The use of TALEN is known to those of skill in the art. In some alternatives described herein, a method of making plasma cells or plasma cell precursors that express a molecule, such as a macromolecule is provided, in which B cells or B cell precursors are subjected to at least one round of genome editing. Methods of genome editing can include, but is not limited to nucleic acid being inserted, deleted or replaced in the genome of a cell. In some alternatives, a nuclease is used to achieve this process. In some alternatives, the nuclease is engineered. In some alternatives, the methods include inducing double strand breaks that are repaired through nonhomologous end-joining (NHEJ) or homologous recombination (HR). In some alternatives, the method includes a first round of genome editing or genome editing. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the Cas nuclease comprises Cas1, Cas2, Cas3, Cas4, Cas5, Cas6, Cas7, Cas8 or Cas9. In some alternatives, the first round of genome editing comprises transducing the B cell with a recombinant adeno-associated virus vector to serve as a donor template for homologous recombination into a candidate genetic loci. In some alternatives, the recombinant adeno-associated virus vector is single-stranded, double stranded or self-complementary.

As used herein, “knock out” includes deleting all or a portion of the target polynucleotide sequence in a way that interferes with the function of the target polynucleotide sequence. For example, a knock out can be achieved by altering a target polynucleotide sequence by inducing an indel in the target polynucleotide sequence in a functional domain of the target polynucleotide sequence (e.g., a DNA binding domain). Those skilled in the art will readily appreciate how to use the CRISPR/Cas systems of the present invention to knock out a target polynucleotide sequence or a portion thereof based upon the details described herein.

### Certain Methods for Preparing a Modified B Cell

Some embodiments of the methods and compositions provided herein include methods for preparing a modified B cell. Some such embodiments include inactivating an endogenous beta-2 microglobulin (B2M) gene in a B cell; and introducing a polynucleotide encoding a fusion protein into the inactivated B2M gene thereby obtaining a modified B cell, wherein the fusion protein comprises an exogenous B2M polypeptide.

In some embodiments, the endogenous B2M gene is inactivated by an insertion, deletion or substitution of at least a portion of the endogenous B2M gene. In some embodiments, each allele of a B2M in a genome, such as a diploid genome is inactivated.

In some embodiments, the inactivating an endogenous B2M gene comprises introducing into the B cell a clustered regularly interspersed short palindromic repeat (CRISPR) nucleic acid coupled to a Cas nuclease. In some embodiments, the Cas nuclease comprises a Cas9 nuclease. In some embodiments, the inactivating an endogenous B2M gene comprises introducing a guide RNA (gRNA) into the B cell. In some embodiments, the gRNA comprises a nucleotide sequence having a sequence identity with the nucleotide sequence of any one of SEQ ID NOs:01-04 that is at least 85%, 86%, 87%, 88%, 89%, 90°/%, 91%, 92%, 93%, 94%, 95%, 96%, 97%, 98%, 99% or 100% or any percentage between any two of the foregoing percentages.

In some embodiments, the inactivating an endogenous B2M gene comprises introducing into the B cell a viral vector comprising a repair template. In some embodiments, the viral vector comprises an adeno-associated virus (AAV) vector.

In some embodiments, the inactivating an endogenous B2M gene comprises introducing a nuclease into the B cell, wherein the nuclease is selected from the group consisting of a zinc finger nuclease, a transcription activator-like effector nuclease (TALEN), a homing endonuclease (HE), and a combined TALEN-HE protein (megaTAL).

In some embodiments, the polynucleotide encoding a fusion protein is introduced into the inactivated B2M gene such that the fusion protein is in-frame with an exon of the endogenous B2M gene.

In some embodiments, the polynucleotide comprises a homology arm, a nucleic acid encoding a self-cleaving peptide, and/or a promoter.

In some embodiments, the introducing a polynucleotide encoding a fusion protein into the inactivated B2M gene introducing into the B cell a CRISPR nucleic acid coupled to a Cas nuclease. In some embodiments, the Cas nuclease comprises a Cas9 nuclease. In some embodiments, the introducing a polynucleotide encoding a fusion protein into the inactivated B2M gene comprises introducing a gRNA into the B cell. In some embodiments, the gRNA comprises a nucleotide sequence having a sequence identity with the nucleotide sequence of any one of SEQ ID NOs:01-04 that is at least 85%, 86%, 87%, 88%, 89%, 90%, 91%, 92%, 93%, 94%, 95%, 96%, 97%, 98%, 99% or 100% or any percentage between any two of the foregoing percentages.

In some embodiments, the introducing a polynucleotide encoding a fusion protein into the inactivated B2M gene comprises introducing into the B cell a viral vector comprising a repair template. In some embodiments, the viral vector comprises an AAV vector.

In some embodiments, the introducing a polynucleotide encoding a fusion protein into the inactivated B2M gene comprises introducing a nuclease into the B cell, wherein the nuclease is selected from the group consisting of a zinc finger nuclease, a TALEN, a HE, and a megaTAL.

In some embodiments, the inactivating an B2M gene in a B cell, and the introducing a polynucleotide encoding a fusion protein into the inactivated B2M gene are performed sequentially. In some embodiments, the inactivating an endogenous B2M gene in a B cell; and the introducing a polynucleotide encoding a fusion protein into the inactivated B2M gene are performed concurrently.

In some embodiments, the fusion protein comprises a therapeutic polypeptide or a therapeutic nucleic acid. In some embodiments, the therapeutic polypeptide comprises an enzyme, an antibody or antigen binding fragment thereof, a receptor, a chimeric antigen receptor, or a cytokine. In some embodiments, the therapeutic polypeptide comprises factor IX, angiotensin-converting enzyme 2 (Ace2), beta-glucocerebrosidase (GBA), alpha-galactosidase A (GLA), or acid alpha-glucosidase (GAA).

More examples of a therapeutic polypeptide or a therapeutic nucleic acid include: (a) affinity reagents designed to inhibit pathogen infections including therapeutic antibodies, immunoadhesins, and bi-specific T cell engaging proteins; (b) immunoinhibitory agents to treat inflammatory conditions, graft rejection and/or autoimmunity including cytokines such as IL10, antibodies such as anti-TNF, and peptides; (c) immune boosting agents designed to increase the host cell response to cancer or pathogens including cytokines, antibodies such as anti-PDL1, and bi-specific T cell engaging proteins; and (d) enzymes designed to cure disease caused by missing proteins including those involved in glycogen storage disorders, or hemophilia such as Factor IX, or Factor VIII.

In some embodiments, the B cell is selected from the group consisting of a hematopoietic stem cell, an early pro-B cell, a late pro-B cells, a large pre-B cell, a small pre-B cell, an immature B cell, a T1 B cell, a T2 B cell, a marginal zone B cell, a mature B cell, a naïve B cell, a plasmablast (short lived) cell, a GC B cell, a memory B cell, and a long lived plasma cell.

### Certain Methods for In Vivo Enrichment of a Modified B Cell

Some embodiments of the methods and compositions provided herein include methods for in vivo enrichment of a modified B cell in a subject, such as an autologous subject. Some such embodiments include inactivating an endogenous B2M gene in a B cell; introducing a polynucleotide encoding a fusion protein into the inactivated B2M gene thereby obtaining a modified B cell, wherein the fusion protein comprises an exogenous B2M polypeptide; and administering the modified B cell to a subject, wherein the B cell is autologous to the subject. In some such embodiments, a modified cell lacking an active endogenous B2M gene, or an exogenous B2M polypeptide has an increased likelihood of clearance by the subject's immune system, such as clearance by natural killer cells.

In some embodiments, the endogenous B2M gene is inactivated by an insertion, deletion or substitution of at least a portion of the endogenous B2M gene. In some embodiments, each allele of a B2M in a genome, such as a diploid genome is inactivated.

In some embodiments, the inactivating an endogenous B2M gene comprises introducing into the B cell a CRISPR nucleic acid coupled to a Cas nuclease. In some embodiments, the Cas nuclease comprises a Cas9 nuclease. In some embodiments, the inactivating an endogenous B2M gene comprises introducing a gRNA into the B cell. In some embodiments, the gRNA comprises a nucleotide sequence having a sequence identity with the nucleotide sequence of any one of SEQ ID NOs:01-04 that is at least 85%, 86%, 87%, 88%, 89%, 90%, 91%, 92%, 93%, 94%, 95%, 96%, 97%, 98%, 99% or 100% or any percentage between any two of the foregoing percentages.

In some embodiments, the inactivating an endogenous B2M gene comprises introducing into the B cell a viral vector comprising a repair template. In some embodiments, the viral vector comprises an AAV vector.

In some embodiments, the inactivating an endogenous B2M gene comprises introducing a nuclease into the B cell, wherein the nuclease is selected from the group consisting of a zinc finger nuclease, a TALEN, a HE, and a megaTAL.

In some embodiments, the polynucleotide encoding a fusion protein is introduced into the inactivated B2M gene such that the fusion protein is in-frame with an exon of the endogenous B2M gene.

In some embodiments, the polynucleotide comprises a homology arm, a nucleic acid encoding a self-cleaving peptide, and/or a promoter.

In some embodiments, the introducing a polynucleotide encoding a fusion protein into the inactivated B2M gene introducing into the B cell a CRISPR nucleic acid coupled to a Cas nuclease. In some embodiments, the Cas nuclease comprises a Cas9 nuclease. In some embodiments, the introducing a polynucleotide encoding a fusion protein into the inactivated B2M gene comprises introducing a gRNA into the B cell. In some embodiments, the gRNA comprises a nucleotide sequence having a sequence identity with the nucleotide sequence of any one of SEQ ID NOs:01-04 that is at least 85%, 86%, 87%, 88%, 89%, 90%, 91%, 92%, 93%, 94%, 95%, 96%, 97%, 98%, 99% or 100% or any percentage between any two of the foregoing percentages.

In some embodiments, the introducing a polynucleotide encoding a fusion protein into the inactivated B2M gene comprises introducing into the B cell a viral vector comprising a repair template. In some embodiments, the viral vector comprises an AAV vector.

In some embodiments, the introducing a polynucleotide encoding a fusion protein into the inactivated B2M gene comprises introducing a nuclease into the B cell, wherein the nuclease is selected from the group consisting of a zinc finger nuclease, a TALEN, a HE, and a megaTAL.

In some embodiments, the inactivating an B2M gene in a B cell; and the introducing a polynucleotide encoding a fusion protein into the inactivated B2M gene are performed sequentially. In some embodiments, the inactivating an endogenous B2M gene in a B cell; and the introducing a polynucleotide encoding a fusion protein into the inactivated B2M gene are performed concurrently.

In some embodiments, the fusion protein comprises a therapeutic polypeptide or a therapeutic nucleic acid. In some embodiments, the therapeutic polypeptide comprises an enzyme, an antibody or antigen binding fragment thereof, a receptor, a chimeric antigen receptor, or a cytokine. In some embodiments, the therapeutic polypeptide comprises factor IX, angiotensin-converting enzyme 2 (Ace2), beta-glucocerebrosidase (GBA), alpha-galactosidase A (GLA), or acid alpha-glucosidase (GAA).

In some embodiments, the B cell is selected from the group consisting of a hematopoietic stem cell, an early pro-B cell, a late pro-B cells, a large pre-B cell, a small pre-B cell, an immature B cell, a T1 B cell, a T2 B cell, a marginal zone B cell, a mature B cell, a naïve B cell, a plasmablast (short lived) cell, a GC B cell, a memory B cell, and a long lived plasma cell.

In some embodiments, the subject is mammalian. In some embodiments, the subject is human.

### Certain Methods for Preparing a Modified B Cell for an Allogeneic Infusion

Some embodiments of the methods and compositions provided herein include methods for preparing a modified B cell for an allogeneic infusion. Some such embodiments include inactivating an endogenous B2M gene in a B cell; and introducing a polynucleotide encoding a fusion protein into the inactivated B2M gene thereby obtaining a modified B cell, wherein the fusion protein comprises: an exogenous B2M polypeptide, a non-polymeric HLA polypeptide, and a self-peptide to be presented by the non-polymeric HLA polypeptide. Some methods also include administering the modified B cell to a subject, wherein the B cell is allogeneic to the subject. In some embodiments, the non-polymeric HLA polypeptide is selected from HLA-E or HLA-G.

In some embodiments, the endogenous B2M gene is inactivated by an insertion, deletion or substitution of at least a portion of the endogenous B2M gene. In some embodiments, each allele of a B2M in a genome, such as a diploid genome is inactivated.

In some embodiments, the inactivating an endogenous B2M gene comprises introducing into the B cell a CRISPR nucleic acid coupled to a Cas nuclease. In some embodiments, the Cas nuclease comprises a Cas9 nuclease. In some embodiments, the inactivating an endogenous B2M gene comprises introducing a gRNA into the B cell. In some embodiments, the gRNA comprises a nucleotide sequence having a sequence identity with the nucleotide sequence of any one of SEQ ID NOs:01-04 that is at least 85%, 86%, 87%, 88%, 89%, 90%, 91%, 92%, 93%, 94%, 95%, 96%, 97%, 98%, 99% or 100% or any percentage between any two of the foregoing percentages.

In some embodiments, the inactivating an endogenous B2M gene comprises introducing into the B cell a viral vector comprising a repair template. In some embodiments, the viral vector comprises an AAV vector.

In some embodiments, the inactivating an endogenous B2M gene comprises introducing a nuclease into the B cell, wherein the nuclease is selected from the group consisting of a zinc finger nuclease, a TALEN, a HE, and a megaTAL.

In some embodiments, the polynucleotide encoding a fusion protein is introduced into the inactivated B2M gene such that the fusion protein is in-frame with an exon of the endogenous B2M gene.

In some embodiments, the polynucleotide comprises a homology arm, a nucleic acid encoding a self-cleaving peptide, and/or a promoter.

In some embodiments, the introducing a polynucleotide encoding a fusion protein into the inactivated B2M gene introducing into the B cell a CRISPR nucleic acid coupled to a Cas nuclease. In some embodiments, the Cas nuclease comprises a Cas9 nuclease. In some embodiments, the introducing a polynucleotide encoding a fusion protein into the inactivated B2M gene comprises introducing a gRNA into the B cell. In some embodiments, the gRNA comprises a nucleotide sequence having a sequence identity with the nucleotide sequence of any one of SEQ ID NOs:01-04 that is at least 85%, 86%, 87%, 88%, 89%, 90%, 91%, 92%, 93%, 94%, 95%, 96%, 97%, 98%, 99% or 100% or any percentage between any two of the foregoing percentages.

In some embodiments, the introducing a polynucleotide encoding a fusion protein into the inactivated B2M gene comprises introducing into the B cell a viral vector comprising a repair template. In some embodiments, the viral vector comprises an AAV vector.

In some embodiments, the introducing a polynucleotide encoding a fusion protein into the inactivated B2M gene comprises introducing a nuclease into the B cell, wherein the nuclease is selected from the group consisting of a zinc finger nuclease, a TALEN, a HE, and a megaTAL.

In some embodiments, the inactivating an B2M gene in a B cell; and the introducing a polynucleotide encoding a fusion protein into the inactivated B2M gene are performed sequentially. In some embodiments, the inactivating an endogenous B2M gene in a B cell, and the introducing a polynucleotide encoding a fusion protein into the inactivated B2M gene are performed concurrently.

In some embodiments, the fusion protein comprises a therapeutic polypeptide or a therapeutic nucleic acid. In some embodiments, the therapeutic polypeptide comprises an enzyme, an antibody or antigen binding fragment thereof, a receptor, a chimeric antigen receptor, or a cytokine. In some embodiments, the therapeutic polypeptide comprises factor IX, angiotensin-converting enzyme 2 (Ace2), beta-glucocerebrosidase (GBA), alpha-galactosidase A (GLA), or acid alpha-glucosidase (GAA).

In some embodiments, the B cell is selected from the group consisting of a hematopoietic stem cell, an early pro-B cell, a late pro-B cells, a large pre-B cell, a small pre-B cell, an immature B cell, a T1 B cell, a T2 B cell, a marginal zone B cell, a mature B cell, a naïve B cell, a plasmablast (short lived) cell, a GC B cell, a memory B cell, and a long lived plasma cell.

In some embodiments, the subject is mammalian. In some embodiments, the subject is human.

### Certain Compositions and Systems

Some embodiments of the methods and compositions provided herein include a modified B cell prepared by any one of the methods provided herein. Some embodiments of the methods and compositions provided herein include a pharmaceutical composition comprising any one of the modified B cells provided herein, and a pharmaceutically acceptable carrier. Some embodiments of the methods and compositions provided herein include an isolated nucleic acid comprising a nucleotide sequence having a sequence identity with the nucleotide sequence of any one of SEQ ID NOs:01-04 of at least 85%, 86%, 87%, 88%, 89%, 90%, 95%, 96%, 97%, 98%, 99%, 100%, or any percentage between any two of the foregoing percentages.

Some embodiments of the methods and compositions include systems for inactivating an endogenous beta-2 microglobulin (B2M) gene in a cell. Some such embodiments include a nuclease capable of cleaving a targeted locus in an endogenous B2M gene in a genome of the cell, or a nucleic acid encoding the nuclease. Some embodiments also include a guide RNA (gRNA) comprising a sequence complementary to the B2M gene, and/or a repair template comprising a first homology arm, a second homology arm, and a nucleic acid encoding a payload therebetween, wherein the first homology arm and/or the second homology arm has homology to a sequence in the B2M gene. In some embodiments, the gRNA is adapted to inactivate the endogenous B2M gene. In some embodiments, the gRNA comprises: a nucleotide sequence having at least 85%, 86%, 87%, 88%, 89%, 90%, 95%, 96%, 97%, 98%, 99%, or 100% sequence identity with the nucleotide sequence of any one of SEQ ID NOs: 01-04; or a variant thereof having no more than 3 mismatches compared to any one of SEQ ID NOs: 01-04. In some embodiments, the targeted locus is within a first coding exon of the B2M gene.

In some embodiments, the nuclease comprises a Cas nuclease. In some embodiments, the Cas nuclease comprises a Cas9 nuclease.

In some embodiments, the nuclease is selected from the group consisting of a zinc finger nuclease, a transcription activator-like effector nuclease (TALEN), a homing endonuclease (HEs), and a combined TALEN-HE protein (megaTALs).

In some embodiments, the repair template lacks a sequence targeted by the nuclease. In some embodiments, the first arm of homology and/or the second arm of homology lack the sequence targeted by the nuclease, and/or lacks a sequence capable of hybridizing to a sequence of the gRNA or complement thereof. In some embodiments, the payload lacks the sequence targeted by the nuclease, and/or lacks a sequence capable of hybridizing to a sequence of the gRNA or complement thereof. In some embodiments, the payload comprises a first nucleic acid encoding a first polypeptide. In some embodiments, the payload comprises a second nucleic acid encoding a second polypeptide. In some embodiments, a nucleic acid encoding a ribosome skip sequence is located between the first nucleic acid and the second nucleic acid.

In some embodiments, the first polypeptide or second polypeptide encodes a B2M cDNA.

In some embodiments, the first polypeptide or second polypeptide encodes a non-polymeric HLA polypeptide. In some embodiments, the non-polymeric HLA polypeptide is selected from HLA-E, HLA-F, or HLA-G. Some embodiments also include a third polypeptide encoding a self-peptide to be presented by the non-polymeric HLA polypeptide.

In some embodiments, the first polypeptide or second polypeptide comprises a therapeutic polypeptide. In some embodiments, the therapeutic polypeptide comprises an enzyme, an antibody or antigen binding fragment thereof, a receptor, a chimeric antigen receptor, or a cytokine. In some embodiments, the therapeutic polypeptide comprises factor IX, angiotensin-converting enzyme 2 (Ace2), beta-glucocerebrosidase (GBA), alpha-galactosidase A (GLA), or acid alpha-glucosidase (GAA).

In some embodiments, the first nucleic acid is operably linked to a promoter of the endogenous B2M gene. In some embodiments, the first polypeptide is in-frame with a polypeptide encoded by the endogenous B2M gene. In some embodiments, the payload comprises a heterologous promoter, and the first nucleic acid is operably linked to the heterologous promoter. In some embodiments, the heterologous promoter is a constitutive promoter. In some embodiments, the promoter selected from an MND promoter, and an EF1 alpha promoter, or a PGK promoter.

In some embodiments, a vector comprises the repair template. In some embodiments, the vector comprises a viral vector. In some embodiments, the viral vector comprises an adeno-associated virus (AAV) vector.

Some embodiments also include a cell, wherein the cell comprises the nuclease; and optionally, the gRNA, and/or repair template. In some embodiments, the cell is selected from the group consisting of a B cell, a hematopoietic stem cell, an early pro-B cell, a late pro-B cells, a large pre-B cell, a small pre-B cell, an immature B cell, a T1 B cell, a T2 B cell, a marginal zone B cell, a mature B cell, a naïve B cell, a plasmablast (short lived) cell, a GC B cell, a memory B cell, and a long lived plasma cell. In some embodiments, the cell is a B cell. In some embodiments, the cell is human. In some embodiments, the cell is autologous to a subject. In some embodiments, the cell is allogeneic to a subject. In some embodiments, the cell is ex vivo. In some embodiments, the cell is in vivo.

Some embodiments include pharmaceutical compositions comprising any one of the systems provided herein and a pharmaceutically acceptable excipient.

Some embodiments include a method for preparing a modified cell, comprising obtaining any one of the systems disclosed herein; and introducing the nuclease into a first cell. Some embodiments also include introducing the gRNA into the cell, and/or introducing the repair template into the cell, thereby preparing a modified cell comprising a modified B2M locus in the genome of the modified cell.

In some embodiments, the modified B2M locus is inactivated. In some embodiments, the gRNA is adapted to inactivate the endogenous B2M gene. Some embodiments also include for the modified cell. In some embodiments, the selecting comprising contacting the modified cell with an immune cell. In some embodiments, the immune cell is allogeneic to the first cell. For example, in some embodiments, the immune cell comprises an MHC-I different from an MHC-I of the first cell. In some embodiments, the immune cell is selected from a T cell, such as a cytotoxic CD8+ T cell, or a natural killer cell. In some embodiments, the immune cell is in vivo. In some embodiments, the immune cell is ex vivo.

In some embodiments, the modified B2M locus expresses an active B2M gene or B2M cDNA. In some embodiments, the repair template lacks a sequence targeted by the nuclease, and/or lacks a sequence capable of hybridizing to a sequence of the gRNA or complement thereof, such that the modified B2M locus lacks the sequence targeted by the nuclease, and/or lacks the sequence capable of hybridizing to a sequence of the gRNA or complement thereof. In some embodiments, the repair template comprises a B2M cDNA. Some embodiments also include selecting for the modified cell. In some embodiments, the selecting comprising contacting the modified cell with an immune cell. In some embodiments, the immune cell is autologous to the first cell. In some embodiments, the immune cell is selected from a T cell, such as a cytotoxic CD8+ T cell, or a natural killer cell. In some embodiments, the immune cell is in vivo. In some embodiments, the immune cell is ex vivo.

In some embodiments, the modified B2M locus expresses a replacement MHC-I. In some embodiments, the repair template comprises a payload encoding a B2M cDNA, a non-polymeric HLA polypeptide, and/or a self-peptide to be presented by the non-polymeric HLA polypeptide. Some embodiments also include selecting for the modified cell. In some embodiments, the selecting comprising contacting the modified cell with an immune cell. In some embodiments, the immune cell is allogeneic to the first cell. In some embodiments, the immune cell is selected from a T cell, such as a cytotoxic CD8+ T cell, or a natural killer cell. In some embodiments, the immune cell is in vivo. In some embodiments, the immune cell is ex vivo. In some embodiments, the first cell is a B cell.

Some embodiments include a method for enriching for a modified B cell having increased resistance to killing by a T cell or natural killer cell, comprising preparing a modified cell by obtaining any one of the systems disclosed herein; and introducing the nuclease into a first cell; and/or introducing the repair template into the cell, thereby preparing a modified cell comprising a modified B2M locus in the genome of the modified cell. In some such embodiments, the first cell is a B cell, and wherein the modified B2M locus is inactivated. Some embodiments also include contacting the modified cell with a T cell or natural killer cell, wherein the T cell or natural killer cell is allogeneic to the first cell, and wherein the modified B cell has an increased resistance to killing by the T cell or natural killer cell compared to a B cell expressing a B2M gene.

Some embodiments include method for enriching for a modified B cell, comprising preparing a modified cell by obtaining any one of the systems disclosed herein; and introducing the nuclease into a first cell; and/or introducing the repair template into the cell, thereby preparing a modified cell comprising a modified B2M locus in the genome of the modified cell. In some embodiments, the modified B2M locus expresses an active B2M gene or B2M cDNA. Some embodiments also include contacting the modified cell with a T cell or natural killer cell, wherein the T cell or natural killer cell is autologous to the first cell, and wherein a cell not expressing an active B2M gene or B2M cDNA is killed by the T cell or natural killer cell.

Some embodiments include method for enriching for a modified B cell, comprising preparing a modified cell by obtaining any one of the systems disclosed herein; and introducing the nuclease into a first cell; and/or introducing the repair template into the cell, thereby preparing a modified cell comprising a modified B2M locus in the genome of the modified cell. In some embodiments, the modified B2M locus expresses a replacement MHC-I, wherein the repair template comprises a payload encoding a B2M cDNA, a non-polymeric HLA polypeptide, and a self-peptide to be presented by the non-polymeric HLA polypeptide. Some embodiments also include contacting the modified cell with a T cell or natural killer cell, wherein the T cell or natural killer cell is allogenic to the first cell.

Some embodiments include a cell prepared by any one of the methods provided herein.

### EXAMPLES

### Example 1—Downregulation of MHC II in Plasma Cells

Major histocompatibility complex (MHC)-I and MHC-II each have a role in graft rejection. Each MHC presents peptide antigens that can trigger immune responses that eventually lead to the demise of the presenting cell. Undifferentiated B cells, and some subsets of differentiated B cells express both MHC-I and MHC-II.

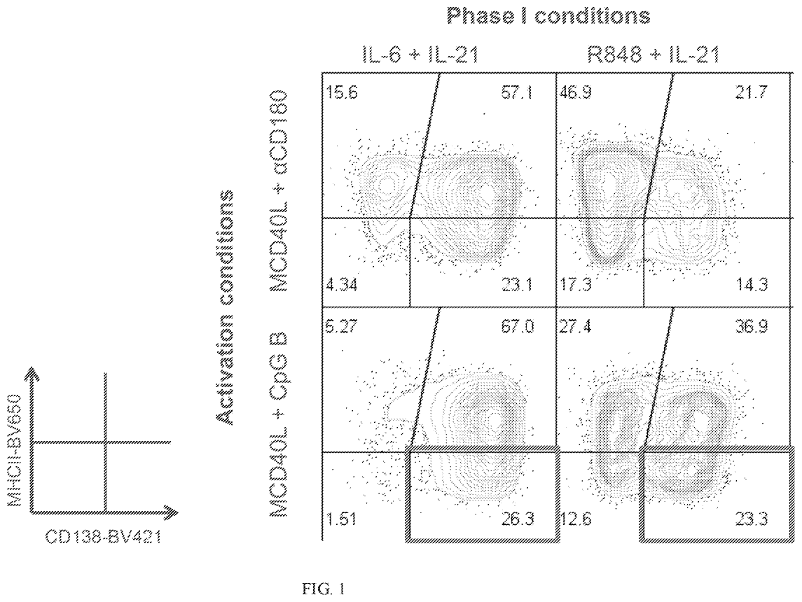

To investigate if differentiated plasma cells down-regulated MHC-II, murine splenic B cells were isolated using negative selection and cultured with various cytokines and feeder cells. Culture conditions included: (1) activation for 2 days with MCD40L and either CpG B or αCD180; (2) phase I for 3 days with IL-21 and either IL-6 or R848; and (3) phase 1H for 3 days with IL-21. Following 8 days of culture, a subset of plasma cells down-regulated MHC-II, as measured using flow cytometry to detect antibodies that bind MHC-II (MHCH-B-BV650). As shown in FIG. 1 conditions including MCD40L and CpG B; and with IL-21 and either IL-6 or R848 resulted in increased percentages (26.3, and 23.3) of cells with reduced levels of MHC-II. Differentiation of B cells into differentiated B cells or a differentiated B cell product using certain culture conditions led to downregulation of MHC-II. The unexpected downregulation of MHC-II in ex vivo differentiated B cell cultures could mitigate allograft rejection that may be invoked by expression of mismatched MHC-II proteins by modified B cells.

### Example 2—Modification of the B2M Locus in B Cells

Both differentiated and undifferentiated B cells express MHC-I and present peptide antigens in MHC-I peptide complexes. The MHC-T-peptide complexes can be directly recognized by T cells that express T cell receptors that have specificity for MHC-T-peptide complexes. Graft rejection can result in instances where a donor MHC-T is different from a recipient MHC-I. Upon T cell recognition of different sequences presented in the MHC-T complexes in the graft, the T cells can directly kill graft cells. MHC-T molecules include a B2M protein, a human leukocyte antigen (HLA) protein, and an antigenic peptide. B2M is required for trafficking of the MHC-I complex to the cell surface.

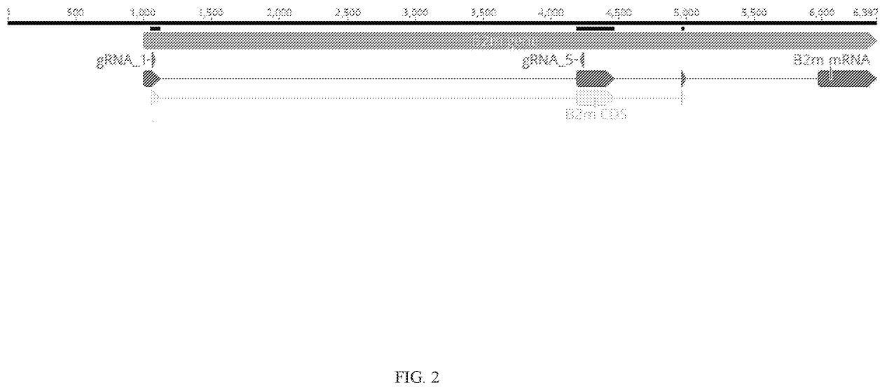

CRISPR targeting of B2M was used to eliminate a functional B2M locus. Two gRNA target sequences (B2M gRNA_1 and B2M gRNA_5) were developed within the coding region of the mouse B2M gene using a ccTop CRISPR/Cas9 target online predictor tool (FIG. 2). gRNA_1 is adjacent to the start codon of the first exon of murine B2M (genome assembly GRCm39/mm39, position 121978227-121978246). gRNA_5 is located in exon 2 of B2M (genome assembly GRCm39/mm39, position 121981407-121981388). Both guides were predicted to result in inactivation/knockout of the endogenous B2M gene via introductions of nucleotide insertions or deletions that introduce frameshifts, or stop codons that render the transcribed RNA susceptible to nonsense mediated decay. B2M gRNA_1 had a proximity to the translation start site and the endogenous B2M promoter which would be advantageous for in-frame insertion of polynucleotides, such as polynucleotides encoding proteins. The sequences for B2M gRNA_1 and B2M gRNA 5 are listed in TABLE 1.

TABLE 1

| BRNA | SEQ ID NO | Sequence |
|---|---|---|
| Murine B2M gRNA_1 | SEQ ID | CTCGGTGACC |
| (gRNA_1) | NO: 01 | CTGGTCTTTC |
| Murine_0B2M gRNA 5 | SEQ ID | TCGGCTTCCC |
| (gRNA_5) | NO: 02 | ATTCTCCGGT |
| Human Guide 1 | SEQ ID | GAGTAGCGCG |
| | NO: 03 | AGCACAGCTA |
| Human Guide 2 | SEQ ID | AAGTCAACTT |
| | NO: 04 | CAATGTCGGA |

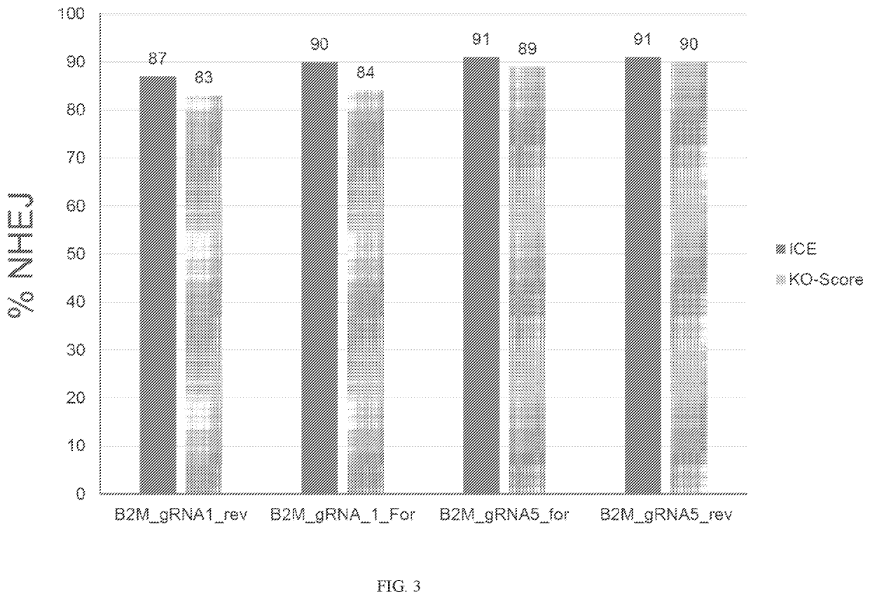

B2M gRNA_1 and gRNA_5, along with recombinant Cas9 were delivered to primary murine B cells as ribonucleoprotein (RNP) complexes. Genomic DNA was prepared from the cells, and a sequence deconvolution analysis was performed. The analysis revealed a high insertion and deletion frequency (ICE score), and high incidence of predicted inactivating indels (KO score) with both guides (FIG. 3).

A substantially similar study was performed in human cells to target the human B2M locus with gRNAs listed in TABLE 1. Human guide_1 is located in the first exon of human B2M (genome assembly GRCh38/hg38, position 44711585-44711566). Human guide 2 is located in exon 2 of human B2M (genome assembly GRCh38/hg38, position 44715531-44715512). Both guides were predicted to result in inactivation/knockout of the endogenous B2M gene via introductions of nucleotide insertions or deletions that introduce frameshifts, or stop codons that render the RNA susceptible to nonsense mediated decay.

### Example 3—Protein Expression in Modified B Cells

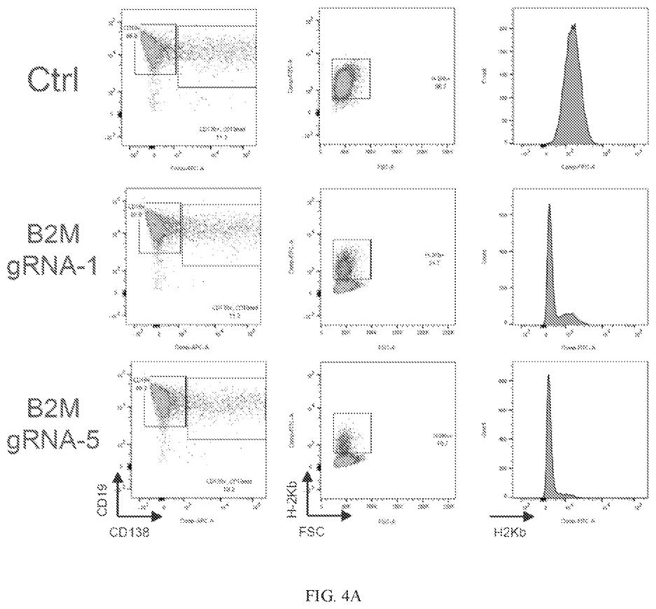

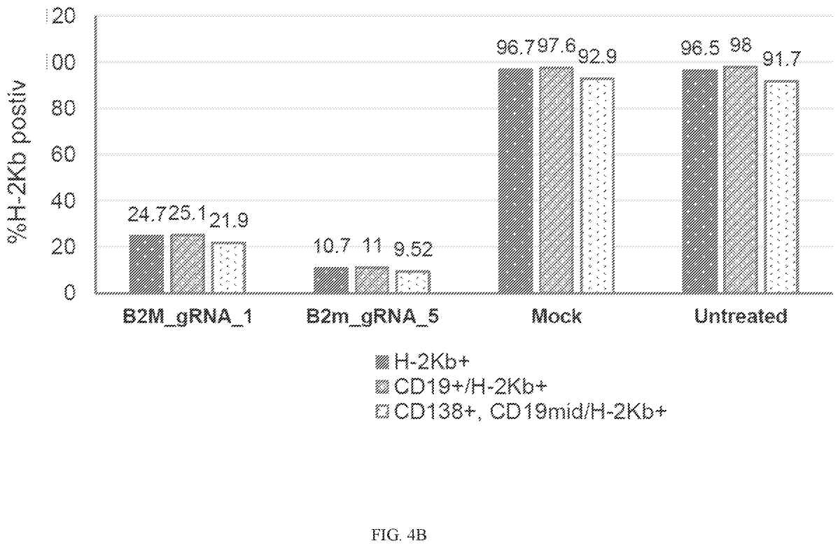

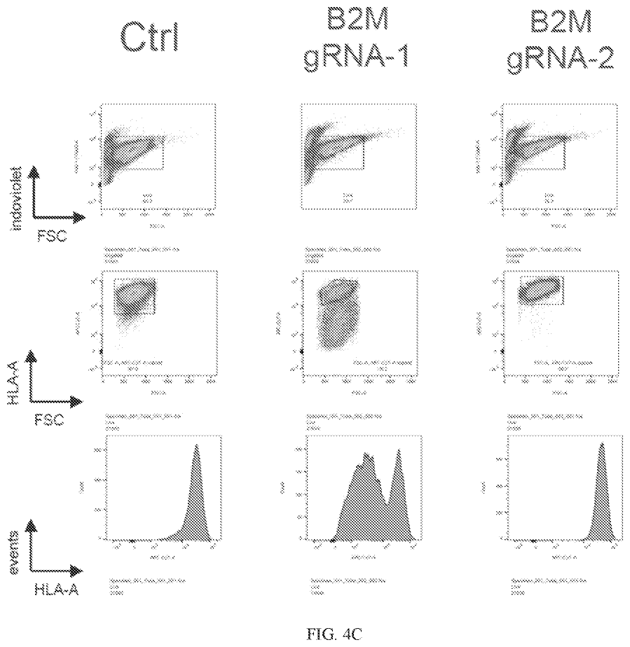

The protein encoded by HLA-B (H2kb) is the primary HLA molecule expressed by C57BL/6 mice. To determine whether B2M knockout eliminated MHC-I expression at the cell surface, H2kb expression in B cells was quantified by flow cytometry. Primary B cell cultures from C57BL/6 mice were treated with B2M gRNA_1 or gRNA_5 RNP complexes. Four days after the treatment, H2Kb protein expression in the cells was analyzed by flow cytometry. The number of H2kb-expressing cells was reduced for cells treated with gRNA_1 or gRNA_5, compared to control (FIG. 4A, FIG. 4B). This flow cytometry-based analysis demonstrated that BM2/MHC-I protein expression was knock-outed/inactivated in greater than 75% of naïve, activated and differentiated B cells: (a) total lymphocytes, (b) CD19+, CD138− B-Cells, and (c) CD19mid, CD138+ plasma cells. Genomic elimination of the functional B2M locus eliminated surface expression of MHC-I complexes.

A substantially similar study was performed in human B cells to target the human B2M locus with gRNAs listed in TABLE 1 (FIG. 4C). Using gRNA-1, but not gRNA-2, expression of MHC-I complexes (HLA-A) was eliminated in greater than 90% of edited B cells. These results are substantially similar to those observed in the B2M depletion experiments in murine B cells.

### Example 4—Functional Analysis of Modified B Cells

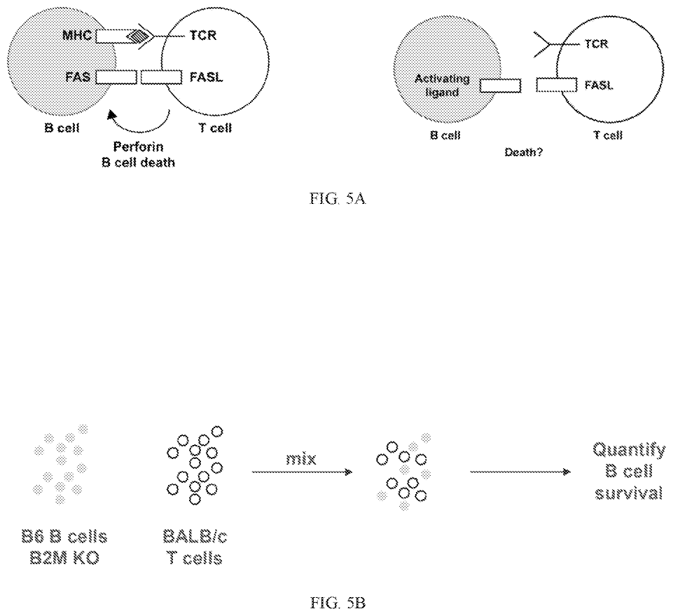

Cytotoxic CD8+ T cells expressing TCRs proteins that recognize mismatched MHC polypeptides kill cells with mismatched MHC-I proteins. However, cytotoxic CD8+ T cells may not kill cells lacking MHC-I, even if the CD8+ T cells and target cells are taken from mismatched donors (FIG. 5A). To determine if B cells lacking MHC-I, by B2M inactivation, would be resistant to killing by such cytotoxic CD8+ T-Cells, a mixed cell assay was performed with T cells from BALB/c mice (HLA, class D) and B cells from C57BL/6 mice (HLA, class B). See FIG. 5B. Briefly, at day 0, Hkd+CD8+ T cells were stimulated with anti-CD3/CD28 beads. At day 3, stimulator beads were removed from the T cells, and the T cells were expanded in IL-2. At day 3, Hkb+ B cells were isolated from spleen and cultured with MCD40L, and CpG. At day 4, the Hkb+ B cells were treated with RNPs to inactivate B2M, or to inactivate an unrelated locus that was used as a negative control (Rosa26), or with mock. At day 6, co-culture groups were established with the treated B cells and the stimulated T-Cells, and treated with MCD40-L, IL-4, IL-2 and no feeder support. At days 7 and 8, all cultures were analyzed by FACS.

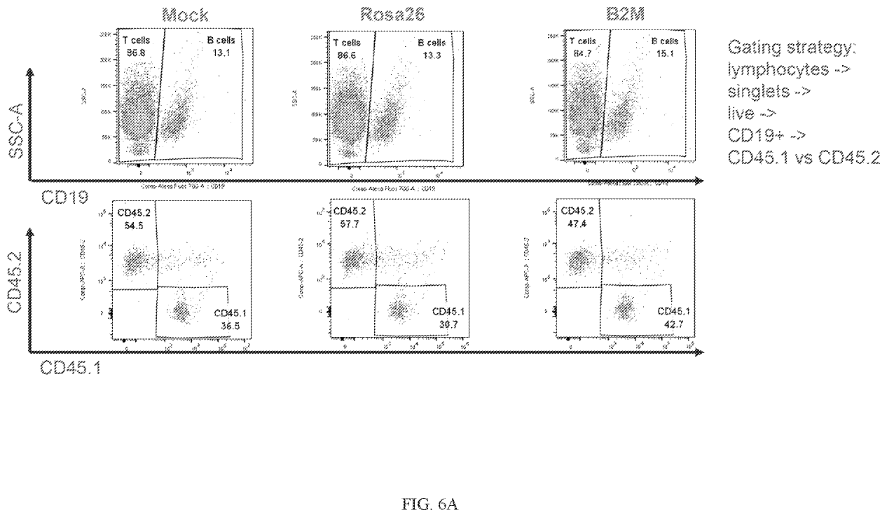

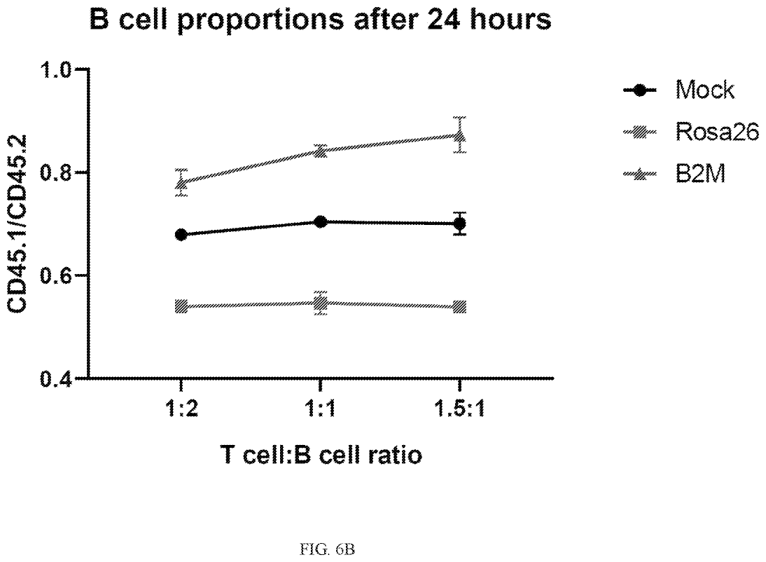

MHC-1 knockout was induced in primary C57BUJ6 (Hkb+) CD45.1 B cells using B2M gRNA_1. These cells were mixed with unedited C57BL/6 CD45.2 B cells and then co-cultured with strain-mismatched Balb/C (Hkd+) CD8+ T cells at various ratios. Populations of B cells treated with B2M gRNA_1 RNP had a higher level of survival and higher levels of CD45.1 expression compared to B cells treated with a Rosa26 RNP or mock (FIG. 6A). After 24 hour co-culture, CD45.1 cells edited with B2M were preferentially represented in the B cell mix relative to mock or Rosa26 edited control cells. B2M knockout cells had an increased survival advantage relative to Rosa26 knockout cells. The ratio of CD45.1 (B2M knockout B cells) to CD45.2 (control B cells) ratios were compared to ratio of T cells to B cells (FIG. 6B). The protective effect of B2M knockout in the CD8 T-Cell killing assay was more pronounced with increasing T cell to B cell ratios. These data showed that genomic elimination of functional B2m protected B cells from T cell killing.

### Example 5—B2M Inactivation Protects Against CD8+ T Cell Killing

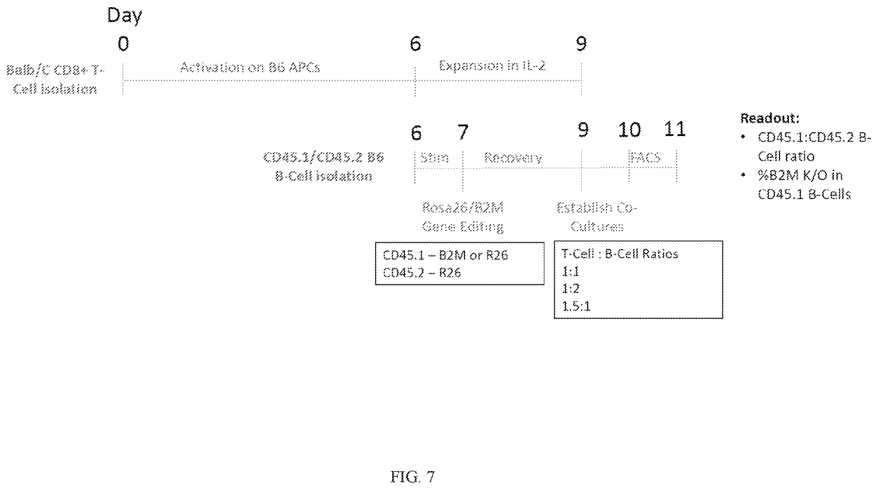

A CD8+ in vitro killing assay was performed. FIG. 7 depicts a timeline for the assay substantially similar to the assay performed in Example 4.

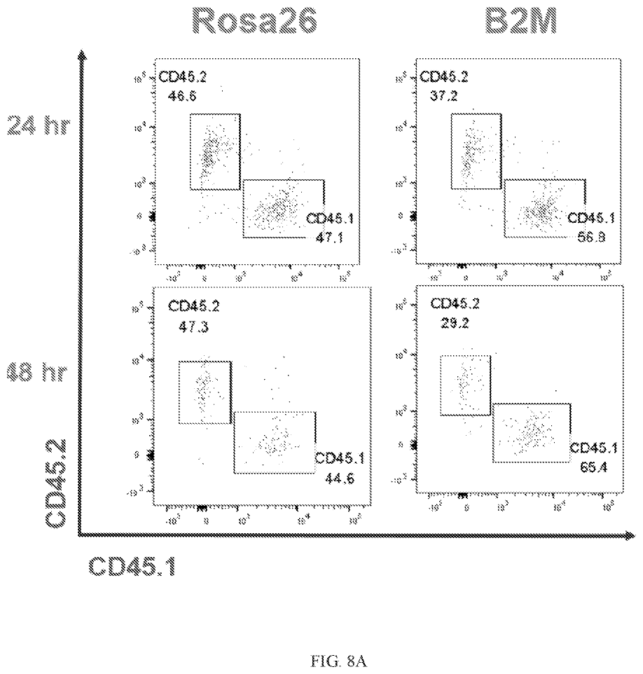

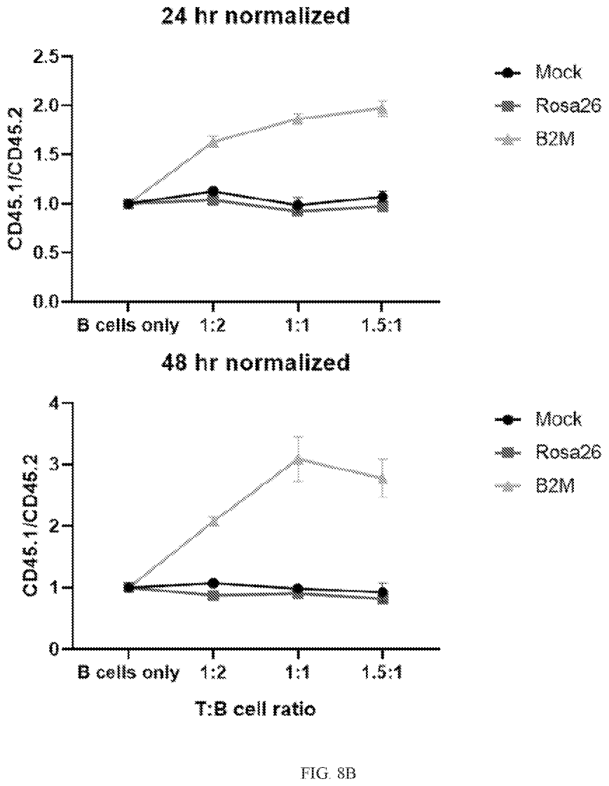

FIG. 8A depicts representative FACS plots for CD45.2 (Target) and CD45.1 (Decoy) C57/B6 B-Cells at 24 h and 48 h Balb/C CD8+ T-Cell co-culture time points. CD45.1 decoy cells were edited with either control RNP targeting the Rosa26 safe harbor locus or B2M RNP to induce MHC1 knockout. FIG. 8A shows that the relative proportions of CD45.2 and CD45.1 cells were stable across both time points in the Rosa26 control, and roughly equivalent to the initial 1:1 Target:Decoy seeding ratio. In contrast, with B2M edited CD42.1 decoy cells, significant skewing toward the decoy population was observed which increased over time, suggesting a protective effect for MHC1 loss.

FIG. 8B depicts quantitation of the CD45.1:CD45.2 ratio from the experiment in FIG. 8A, normalized to the base ratio in co-culture conditions with no CD8+ T-Cells. The ratios were plotted for the 24 h co-culture time point (upper panel) and 48 h co-coculture time point (lower panel) across increasing CD8+ T-Cell to B-Cell ratios. FIG. 8B shows that the CD45.1:CD45.2 ratio was stable at both time points for Mock edited or Rosa26 control edited CD45.1 decoy B-Cells, but progressively increased with increased CD8+ T-Cell ratio for B2M CD45.1 decoy cells. N=3 for each data point.

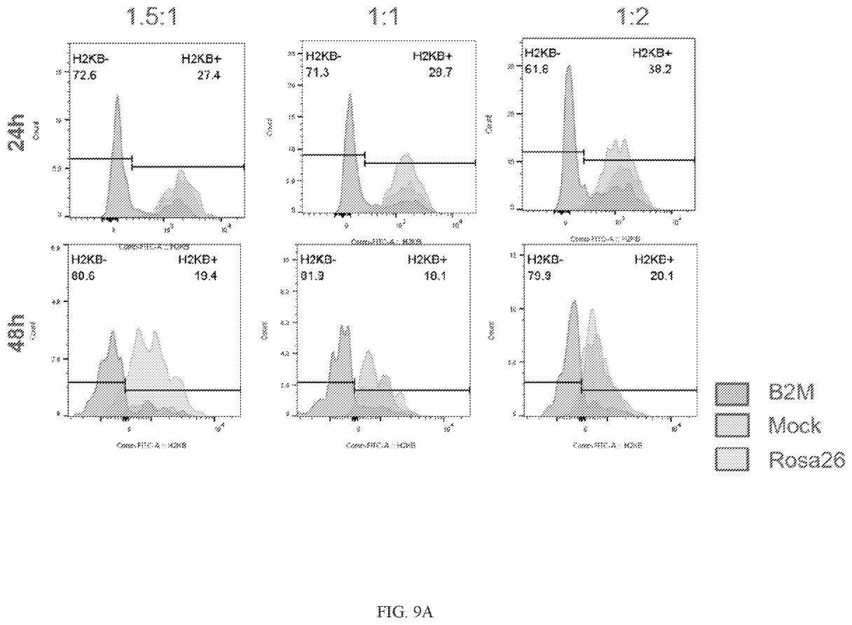

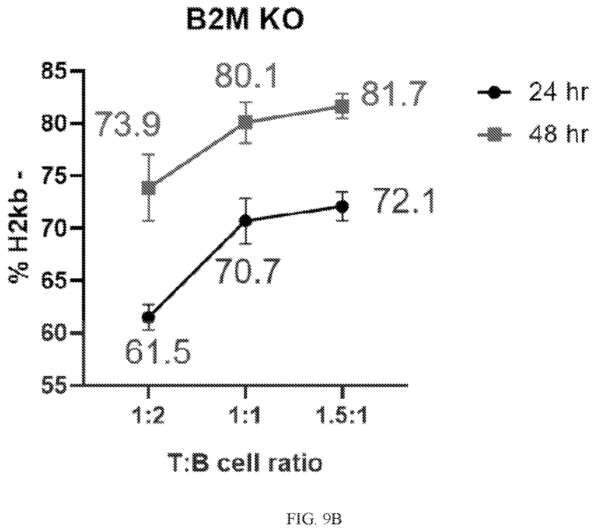

FIG. 9A depicts a representative FACS analysis for H2Kb (the relevant MHC1 haplotype in C57/B6 B-Cells) within the CD45.1 B-Cell population in the experiment related to FIG. 8A. The 24 h co-culture time point (upper row) and 48 h co-coculture time point (lower row) are shown with decreasing CD8+ T-Cell to B-Cell ratios of 1.5:1, 1:1 and 1:2. Each histogram overlays the plots for the three different CD45.1 decoy populations: Mock Edited, Rosa26 Control Edited, and B2M edited. The percentage values shown in each histogram are for the B2M edited group. FIG. 9A shows that the level of MHC1 knockout in the B2M edited group increased both over time and with increasing CD8+ T-Cell ratio, suggesting selection for this MHC1 negative population in the presence of cytotoxic CD8+ T-Cells.

FIG. 9B depicts a quantitation of H2Kb knockout level in B2M edited CD45.1 B-Cells at 24 h and 48 h time points with increasing CD8+ T-Cell ratios. FIG. 9B shows that there was a progressive increase in MHC1 knockout percentage with increasing CD8+ T-Cell ratio at both time points. N=3 for each data point.

### Example 6—Restoration of B2M Expression in B Cells

NK cells perform immune surveillance to eliminate potentially harmful cells that do not express MHC-I molecules on the surface. One possible application of engineering the B2M locus is to introduce cargo DNA into the B2M locus using CRISPR and homology directed repair and use the natural immune surveillance function of NK cells to enrich for engineered cell populations. In a homology-directed repair experiment, CRISPR gRNAs are used to disrupt the genomic target locus, and AAV-delivered repair templates are incorporated into the locus, replacing the endogenous sequence. In typical experiments, the genomic disruption event is highly efficient, and the homology-directed repair event is less efficient. The resulting cell population contains a majority of cells that have only knock-out alleles and a smaller subset that express repaired alleles.

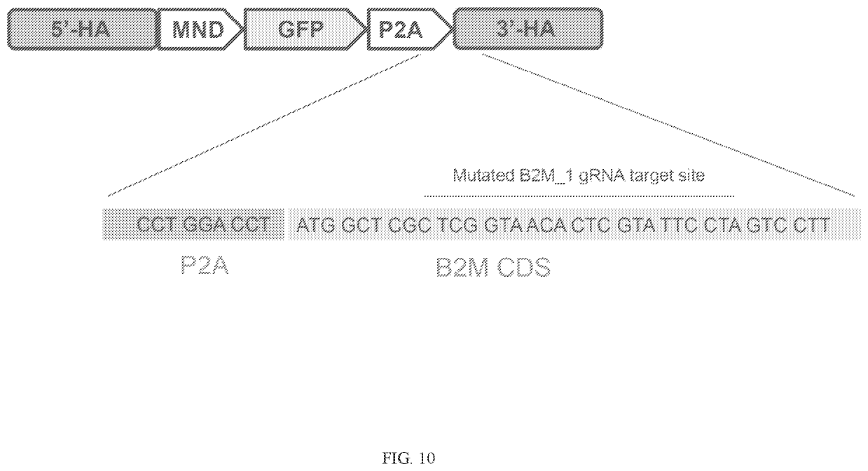

In this example, a construct including an HDR donor template was developed to modify the B2M locus to include a strong MND promoter and a GFP marker, such that the resulting sequence would be in-frame with the endogenous B2M coding sequence (FIG. 10). The construct contained a mutated version of the B2M gRNA_1 target site. A P2A self-cleaving sequence separated the GFP from B2M cDNA, allowing expression of a trackable marker in HDR-edited cells that also expressed artificially reconstituted B2M coding sequence. This donor template (designated 3307), was designed to be delivered as an adeno associated virus (AAV).

In a B cell in which the B2M locus had been previously knocked-out/inactivated, successful modification of the B2M locus with the construct should restore MHC-I expression to the B cell. Conversely, in a B cell in which the B2M locus had been previously knocked-out/inactivated, unsuccessful modification of the B2M locus with the construct, for example by improper insertion at a non-B2M locus or an improper partial insertion, would not restore MHC-I expression to the B cell. Briefly, partially edited cells are H2Kb−; these should die in vivo due to NK cell surveillance; H2Kb should be restored in GFP+ cells; and only HDR and unedited cells should survive in vivo.

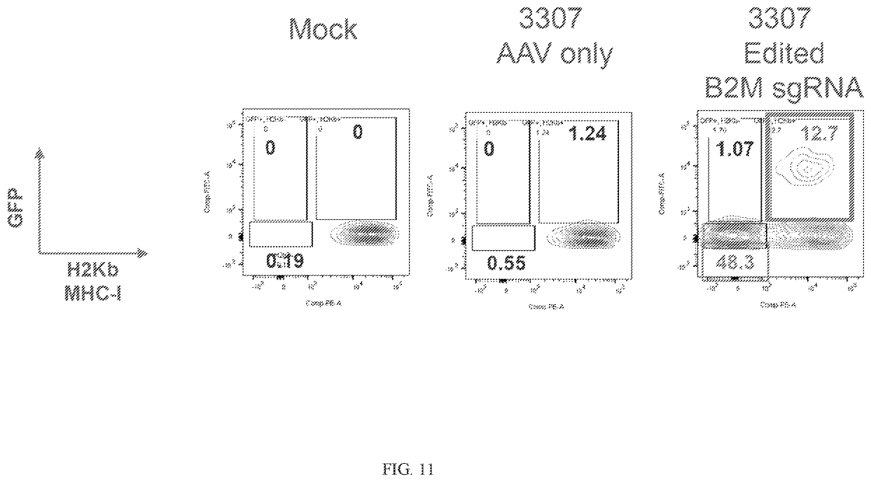

Homology-directed repair in primary mouse B-Cells was elicited using a combination of B2M gRNA_1 RNP delivery and 3307 AAV transduction. In cells receiving both RNP and virus, a population of GFP+ cells was observed 3 days post-editing that was also MHC-I positive (FIG. 11). No GFP+ cells were observed in the MHC-1 negative population. Cells receiving the 3307 virus construct without RNP were entirely GFP negative, which was expected since AAV episomes would not be stable in cycling B cells, and the DNA sequence would not have integrated without co-delivery of the associated B2M CRISPR reagents. This demonstrated that the B2M locus could be successfully modified and selected in vivo for cells in which (1) no modification has occurred; and (2) a portion of the B2M locus had been modified such that the locus was resistant to inactivation with the gRNA, and expressed a B2M product.

### Example 7—Survival of B Cells with Restored B2M cDNA Expression

B cells are prepared according to Example 6 in which the endogenous B2M locus is knocked-out/inactivated, and an exogenous coding sequence is inserted in-frame, along with the B2M cDNA at the B2M locus.

A population of the cells prepared from a subject is administered to the same subject, or a different subject with a matched MHC-I sequence. Prepared cells in which the construct properly modified the B2M locus, the prepared cells express B2M cDNA at the B2M locus and are resistant to NK cell killing in vivo. In contrast, prepared cells in which the B2M locus is not properly modified by the construct, and do not express the B2M cDNA at the B2M locus are sensitive to NK cell killing in vivo, and are cleared by NK cells in vivo. In some embodiments, a similar strategy can be used to select for engineered B cells in vivo, for example with an engineered B cell autologous to a subject.

In another example, a B cell is obtained from a first subject. The cell is modified. The modified cell is administered to the first subject, or to a second subject having a MHC-I sequence matching with a MHC-I sequence of the first subject. The cell is modified at a B2M genomic locus by insertion of a B2M cDNA into the B2M genomic locus. Modified cells expressing the B2M cDNA are resistant to NK cell killing in vivo. In contrast, cells which do not express the B2M cDNA are sensitive to NK cell killing in vivo, and are cleared by NK cells in vivo.

In another example, B cells are prepared according to Example 5 in which endogenous B2M and/or CIITA is knocked-out/inactivated, and an exogenous coding sequence is inserted to replace B2M. In some embodiments, the B2M locus is modified to express (a) a trimer including an engineered HLA-E, a common self-peptide that can inactivate NK cells, and B2M; (b) CD47, a surface protein that can inhibit NK cell cytotoxic activity; or (c) CD24, a surface protein that can inhibit NK cell activity.

### Example 8-Substitution of B2M Locus in B Cells

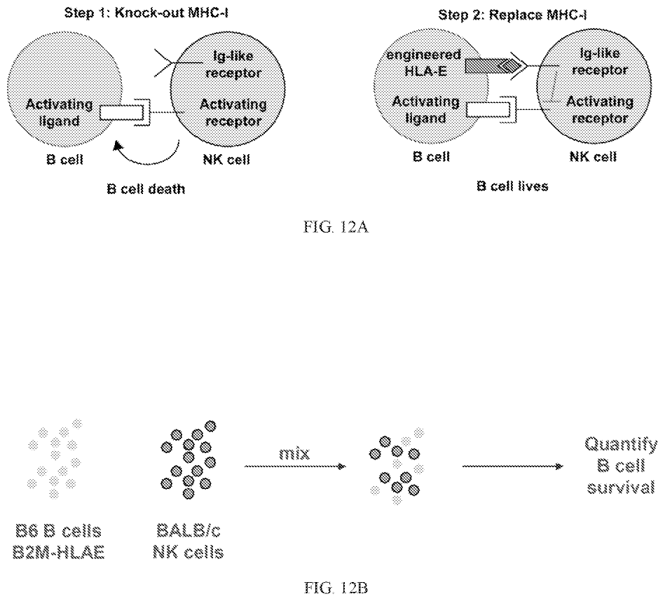

HLA molecules are highly polymorphic, meaning that between different people HLA molecules have many different sequences. Within MHC-I, a person expresses one type of HLA. HLA-A, HLA-B and HLA-C are considered highly polymorphic, whereas HLA-E, HLA-F, and HLA-G are less polymorphic. A strategy for a substitution at the B2M locus is depicted in FIG. 12A in which the B2M locus is modified to inactivate endogenous B2M expression, and to express a trimer consisting of engineered HLA-E, a common self-peptide that is not typically mutated in people, and B2M. After reconstitution of the B2M locus with this nonpolymorphic trimer, cells express the trimer at the cell surface, but not express the endogenous, highly polymorphic HLA. B cells expressing this engineered trimer taken from mismatched individuals do not elicit CD8+ T cell killing but inhibit NK cell immune surveillance. A mixed cell assay to test B cell survival of a modified B cell is depicted in FIG. 12B.

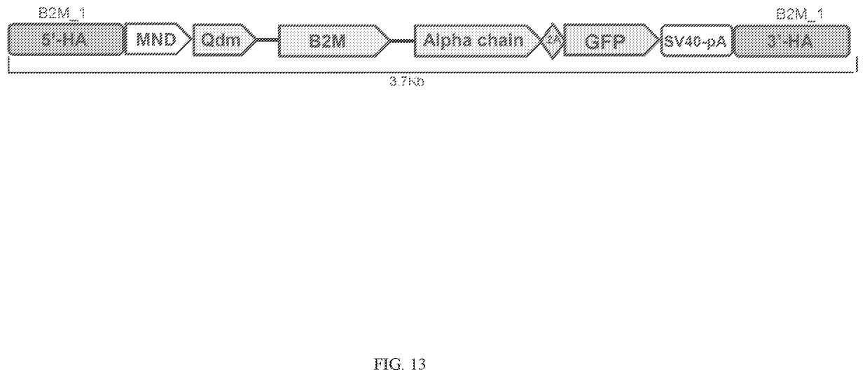

A construct including an HDR donor template was developed to deliver a polynucleotide encoding a non-polymorphic HLA-E fusion polypeptide containing a Qdm self-peptide, B2M, and an HLA-E alpha chain connected via flexible linker sequences and driven by an exogenous MND promoter (FIG. 13). The Qdm was a self-peptide presented by the non-polymorphic HLA-E. The fusion polypeptide was paired to a GFP fluorophore for tracking and was designed to induce endogenous B2M knockout. The construct (designated 3310) containing the repair template for the trimer was designed to be delivered as an adeno associated virus (AAV). Example nucleotide sequences for construct 3210 are listed in TABLE 2.

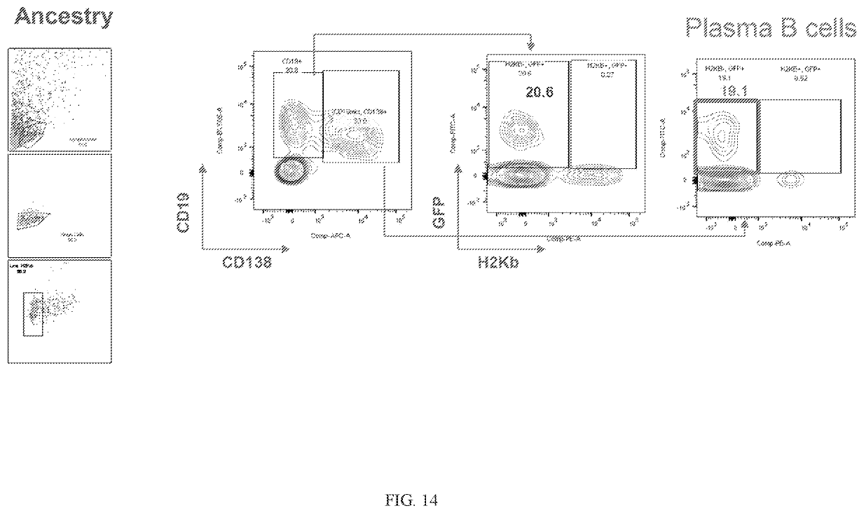

Primary mouse B-Cells isolated from C57BL/6 mice were edited using a combination of B2M gRNA_1 RNP delivery and 3310 AAV transduction. In cells that underwent homology-directed repair, GFP was detectable in both CD19+, CD38− B-Cells and CD19mid, CD138+ plasma cells at 7 days post-editing (FIG. 14). Importantly, in both populations GFP expression was completely co-incident with loss of HLA-B (Hk2b) surface expression, while HLA-B positive cells were entirely GFP negative, suggesting that the HLA-E fusion protein had replaced endogenous MHC-I expression in these cells.

TABLE 2

| Feature | |
|---|---|
| (SEQ ID NO) | Sequence |
| 5′ Homology arm | CACCAAACTCAAGAGCACACCCTAGATAGTAGGGCACCAA |
| (SEQ ID NO: 05) | GGGTCCAGCCCAGGCTGTTTGAAATATCACGGGACTTTATA |
| | AGAACATGAAACTGAAAATGGGAAAGTCCCTTTGTAACCT |
| | AGTTCAGCATCAACAGCTAGGAGACTGGTGACGACCTCCG |
| | GATCTGAGTCCGGATTGGCTGTGAGTTCAGGAACTATATAA |
| | GAGCGCGCGCCCTGGCTGGCTCTCATTTCAGTGGCTGCTAC |
| | TCGGCGCTTCAGTCGCGGTCGCTTCAGTCGTCAGCATGGCT |
| | CGCTCGGTGACCCTGGT |
| MND promoter | GAACAGAGAAACAGGAGAATATGGGCCAAACAGGATATC |
| (SEQ ID NO: 06) | TGTGGTAAGCAGTTCCTGCCCCGGCTCAGGGCCAAGAACA |
| | GTTGGAACAGCAGAATATGGGCCAAACAGGATATCTGTGG |
| | TAAGCAGTTCCTGCCCCGGCTCAGGGCCAAGAACAGATGG |
| | TCCCCAGATGCGGTCCCGCCCTCAGCAGTTTCTAGAGAACC |
| | ATCAGATGTTTCCAGGGTGCCCCAAGGACCTGAAATGACC |
| | CTGTGCCTTATTTGAACTAACCAATCAGTTCGCTTCTCGCTT |
| | CTGTTCGCGCGCTTCTGCTCCCCGAGCTCTATATAAGCAGA |
| | GCTCGTTTAGTGAACCGTCAGATC |
| Qdm peptide | GCTATGGCTCCCAGGACACTCCTGCTG |
| (SEQ ID NO: 07) | |
| Beta-2 | ATTCAGAAGACACCTCAAATCCAGGTGTACTCCAGACACC |
| microglobulin | CCCCCGAGAACGGAAAGCCTAATATTCTCAATTGTTACGTG |
| (SEQ ID NO: 08) | ACCCAGTTTCACCCCCCCCATATTGAGATCCAGATGCTCAA |
| | AAACGGCAAGAAAATTCCTAAGGTGGAGATGAGCGACATG |
| | AGCTTTAGCAAGGACTGGAGCTTCTACATTCTCGCCCACAC |
| | AGAATTTACACCCACCGAGACCGACACCTATGCTIGCAGG |
| | GTGAAACATGCCAGCATGGCTGAGCCTAAGACAGTGTACT |
| | GGGATAGGGACATG |
| Alpha chain | AGCCCACACTCGCTGCGGTATTTCACCACCGCCGTGTCCCG |
| (SEQ ID NO: 09) | GCCCGGCCTCGGGGAGCCCCGGTTCATCATTGTCGGCTACG |
| | TGGACGACACGCAGTTCGTGCGCTTCGACAGCGACGCGGA |
| | AAATCCGAGGATGGAGCCTCGGGCGCGGTGGATTGAGCAG |
| | GAGGGGCCGGAGTATTGGGAGCGGGAGACTTGGAAAGCC |
| | AGGGACATGGGGAGGAACTTCAGAGTAAACCTGAGGACCC |
| | TGCTCGGCTACTACAATCAGAGTAACGACGAATCTCACAC |
| | GCTGCAGTGGATGTACGGCTGCGACGTGGGGCCCGATGGG |
| | CGCCTGCTCCGCGGGTATTGTCAGGAGGCCTACGATGGCC |
| | AGGATTACATCTCCCTGAACGAGGACCTGCGTTCCTGGACC |
| | GCGAATGACATAGCCTCACAGATCTCTAAGCACAAGTCAG |
| | AGGCAGTCGATGAGGCCCACCAACAGAGGGCATACCTGCA |
| | AGGTCCTTGCGTGGAGTGGCTCCATAGATACCTACGGCTGG |
| | GAAATGAGACACTGCAGCGCTCAGACCCTCCAAAGGCACA |
| | TGTGACCCATCACCCTAGATCTGAAGATGAAGTCACCCTGA |
| | GGTGCTGGGCCCTGGGCTTCTACCCTGCTGACATCACCCTG |
| | ACCTGGCAGTTGAATGGGGAGGAGCTGACCCAGGACATGG |
| | AGCTTGTGGAGACCAGGCCTGCAGGGGATGGAACCTTCCA |
| | GAAGTGGGCAGCTGTCGTGGTGCCTCTTGGGAAGGAGCAG |
| | TATTACACATGCCATGTGTACCATGAGGGGCTGCCTGAGCC |
| | CCTCACCCTGAGATGGGAGCCTCCTCCATCCACTGTCTCCA |
| | ACATGGTAATCATAGCTGTTCTGGTTGTCCTTGGAGCTGTG |
| | ATCATCCTTGGAGCTGTGGTGGCTTTTGTGATGAAGAGGAG |
| | GAGACACATAGGIGTAAAAGGATGCTATGCTCATGTTCTA |
| | GGCAGCAAGAGCTTCCAGACCTCTGACTGGCCTCAGAAGG |
| | CA |
| 3′ Homology arm | TTCTGGTGCTTGTCTCACTGACCGGCCTGTATGCTATCCAG |
| (SEQ ID NO: 10) | AGTGAGTGCCTCTTICCCCTCTCTTGGCATTAAATTTTTAGT |
| | TCTCCTTAGTTCTCCTTCCCGACGGGATTGGCCAGGGCTGC |
| | GTCTCTCGGGAAAGGAGTGGGCGTCCCGCTGCTTACAGCTT |
| | TGAGGCTACAGGGTGGATGCGCCTGTTTGCGAGTGTGCTG |
| | ACGGTCAGCGCTGAAGATGGGGAAGGCTTCTCTTTTTCTCC |
| | TCTGCTGGCGGGAGCCCCTAGACTCCCTGAGCGCAAAAGG |
| | AGGGTGGGGCTGCCT |

### Example 9—In Vivo Survival of Allografted Gene-Edited Stealth B Cells

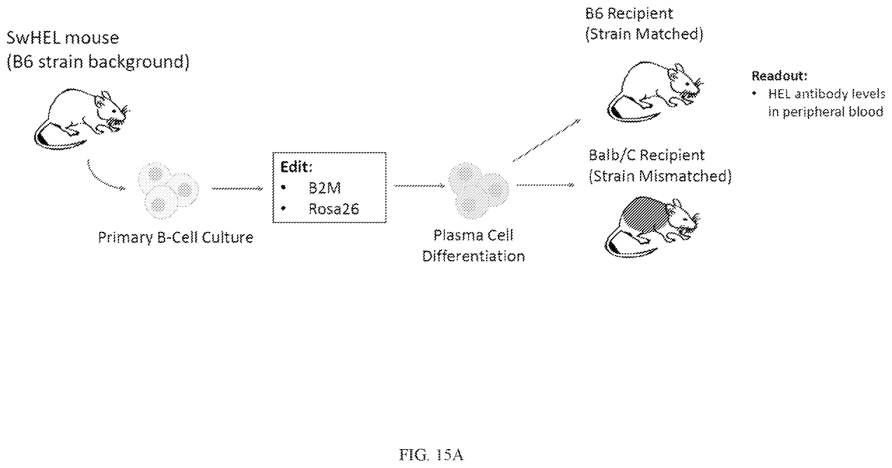

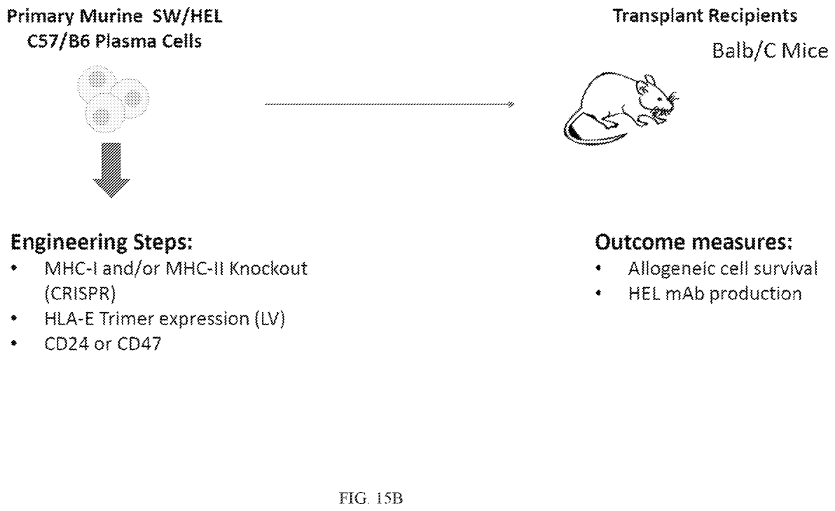

To establish parameters for NK killing, an in vivo study performed substantially similar to the study depicted in FIG. 15A and FIG. 15B is performed. Primary mouse B cells are isolated from C57BL/6 mice expressing a fixed B cell receptor/antibody (SW-HEL). The cells are modified as described in Example 6 and 8 such that modified cells express the HLA-E trimer, CD47, CD24, or a combination containing the HLA-E trimer with CD47 or CD24. Following expansion of the modified cells on feeder cells expressing murine BAFF, murine CD40L and supplemented with soluble R848 or murine IL21, about 10 million modified cells are transferred into BaIB/C mice. Following cell transfer, engraftment is tracked by measuring SW-HEL antibody production over time. Engraftment is compared with C57BL/6 cells edited to express GFP in the B2M locus and with transfers of all cells from all editing strategies into C57BL/6 mice. Allogeneic B cells expressing HLAE-trimers, CD47, CD24 or a combination thereof express SW-HEL following transfer into mis-matched recipient animals. Detection of antibody expressed by the transferred cells indicates successful engraftment. In contrast, cells engineered to express GFP and not B2M, or cells edited to delete B2M do not survive transfer due to clearance by NK cells. Cells that are unedited do not engraft successfully due to clearance by T cells. Successful replacement of MHC class 1 with HLAE trimers, CD47 and/or CD24 demonstrates that modified B cell populations survive upon transfer into mis-matched recipients.

### Example 10− In Vivo Survival of Allografted Gene-Edited Stealth B Cells

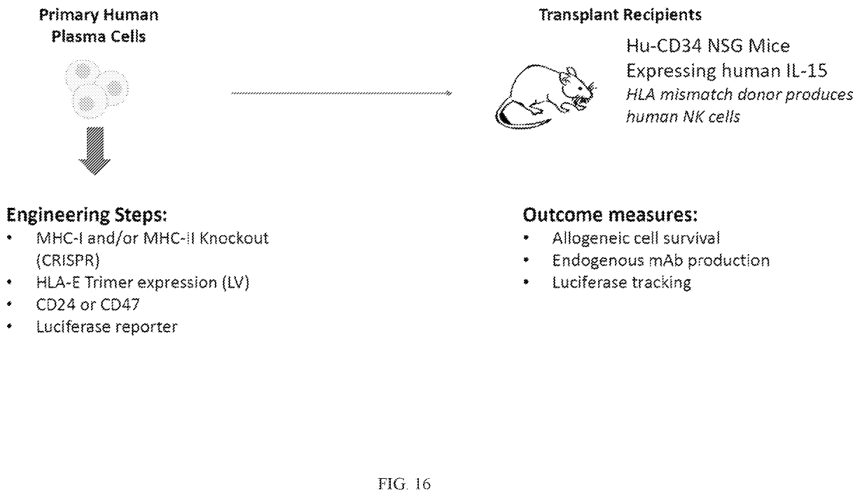

A study substantially similar to a study depicted in FIG. 16 is performed. Human B cells are isolated from peripheral blood mononuclear cells and are edited such that modified cells express the HLA-E trimer, CD47, CD24, or combination thereof containing the HLA-E trimer with CD47 or CD24. The modified cells are expanded and differentiated in vitro using a three-phase differentiation procedure such as disclosed in U.S. 2018/0282692 which is incorporated by reference in its entirety. Following expansion and differentiation, 10 million engineered cells are transferred into immunodeficient mice (NSG) engineered to express IL15 and previously engrafted with human CD34 cells isolated from a different, mismatched donor. NSG-IL15 mice humanized via transfer with human CD34 cells are capable of developing NK cells, enabling assessment of NK cell killing. Following cell transfer, engraftment is tracked by measuring human IgG antibody production over time. NSG mice humanized with CD34 cells do not produce IgG antibody. Engraftment of allogeneic edited cells is compared with B cells edited to express GFP in the B2M locus and with transfers of all cells from all editing strategies into autologous humanized NSG-IL15 mice.

Allogeneic B cells expressing HLAE-trimers, CD47, CD24 or a combination thereof express hIgG following transfer into mis-matched recipient animals. Detection of antibody expressed by the transferred cells indicates successful engraftment. In contrast, cells engineered to express GFP and not B2M, or cells edited to delete B2M do not survive transfer due to clearance by NK cells. Cells that are unedited do not engraft successfully due to clearance by T cells. Successful replacement of MHC class 1 with HLAE trimers, CD47 and/or CD24 demonstrates modified B cell populations survive upon transfer into mis-matched recipients.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A system for modifying an endogenous beta-2 microglobulin (B2M) gene in a cell, comprising:
a nuclease capable of cleaving a targeted locus in an endogenous B2M gene in a genome of the cell, or a nucleic acid encoding the nuclease; and optionally:
a guide RNA (gRNA) comprising a sequence complementary to the B2M gene, and/or
a repair template comprising a first homology arm, a second homology arm, and a nucleic acid encoding a payload therebetween, wherein the first homology arm and/or the second homology arm has homology to a sequence in the B2M gene.

2-77. (canceled)
